(19) United States
(12) Patent Application Publication
(10) Pub. No.: US 2026/0159591 A1
(43) Pub. Date: Jun. 11, 2026

(54) ANTIBODIES THAT BIND TO INTEGRIN ALPHA11 AND USES THEREOF

(71) Applicant: Fibrocor Therapeutics Inc., Toronto (CA)

(72) Inventors: William C. FANSLOW, Normandy Park, WA (US); Nuno Miranda Guerra COELHO, Toronto (CA); James Andrew THOMPSON, Cambridge (GB)

(21) Appl. No.: 19/123,491

(22) PCT Filed: Oct. 20, 2023
(86) PCT No.: PCT/CA2023/051397

§ 371 Date: Apr. 23, 2025

Related U.S. Application Data
(60) Provisional application No. 63/380,668, filed on Oct. 24, 2022.

(51) Int. Cl. C07K 16/28

(52) U.S. Cl. CPC C07K 16/2839; C07K 2317/622; C07K 2317/76; C07K 2317/92

(57) ABSTRACT

The present disclosure provides antibodies or antigen-binding fragments thereof that bind specifically to integrin all (ITGA11). This disclosure also provides methods for treating a disorder in a subject, such as a fibrotic disorder, an inflammatory disorder, or a cancer. This disclosure includes related pharmaceutical compositions, polynucleotides, vectors, host cells, methods of production, methods of treatment, diagnostic methods, and kits.

### RELATED APPLICATIONS

This Patent Cooperation Treaty application claims the benefit of priority of U.S. Provisional Application No. 63/380,668 filed on Oct. 24, 2022, which is incorporated herein by reference in its entirety.

### BACKGROUND

Interactions between cells and their extracellular microenvironment are primarily mediated by a family of cell surface receptors known as integrins, which are recognized as key molecules involved in myofibroblast differentiation. Integrins are heterodimeric transmembrane receptors composed of a and B subunits that can combine to form 24 different integrin heterodimers. Integrins regulate cytoskeletal dynamics, thereby influencing a number of crucial cellular processes, for example, cell adhesion, migration and differentiation. Integrins also have a key role in the activation of growth factors such as transforming growth factor beta (TGFβ). Integrin alpha chain α11 (ITGA11) interacts with integrin beta chain β1 to form the α11β1 heterodimer. α11β1 preferentially binds to type I collagen and has been shown to be expressed on cultured embryonic fibroblasts, cardiac fibroblasts and in activated liver, lung and renal myofibroblasts. α11β1 has been shown to be upregulated by TGFß and regulates embryonic mesenchymal cell differentiation on the collagen matrix. α11β1 has also been implicated in inducing tumor growth and the metastatic potential of small-cell lung carcinoma cells. ITGA11 is overexpressed in fibrotic disease tissues, including the lungs of IPF patients and the kidney of patients with chronic allograft nephropathy Accordingly, ITGA11 is a target for therapeutic intervention and there is a need for therapeutic modalities that bind to ITGA11.

### SUMMARY

The present disclosure provides antibodies or antigen-binding fragments thereof that bind specifically to integrin α11 (ITGA11). The disclosure also provides methods for treating an ITGA11-associated disorder in a subject, such as a fibrotic disorder, an inflammatory disorder, or a cancer. Also included in the disclosure are related pharmaceutical compositions, polynucleotides, vectors, host cells, methods of production, methods of treatment, diagnostic methods, and kits.

In one aspect, the disclosure provides an antibody or an antigen-binding fragment thereof that binds specifically to integrin α11 (ITGA11), wherein the antibody or antigen-binding fragment thereof includes:
a complementarity-determining region (CDR) heavy chain 1 (CDR-H1) including the amino acid sequence of GFTFSSYA (SEQ ID NO: 1), GFTFSNAW (SEQ ID NO: 9), GFTFSSYS (SEQ ID NO: 14), GYTFTDYY (SEQ ID NO: 28), GFTFSDYW (SEQ ID NO: 36), or GFMFDTHA (SEQ ID NO: 46);
a complementarity-determining region (CDR) heavy chain 2 (CDR-H2) including the amino acid sequence of ISGSGGST (SEQ ID NO: 2), ISSSSSTI (SEQ ID NO: 15), FDPEDGET (SEQ ID NO: 29), or ISGSGGSI (SEQ ID NO: 74);
a complementarity-determining region (CDR) heavy chain 3 (CDR-H3) including the amino acid sequence of AKDLDWSGHDAFDI (SEQ ID NO: 3), ARDRGYSYSETSNDAFDI (SEQ ID NO: 10), ARGPDLSDYFDY (SEQ ID NO: 16), AKDPRGSGRDDAFDI (SEQ ID NO: 20), AKDPTTMTTDAFDI (SEQ ID NO: 25), ATLDYRGVVYFDY (SEQ ID NO: 30), AKDLLWAARDAFDI (SEQ ID NO: 37), AKQTVTSADDYFDY (SEQ ID NO: 43), ARSGETAGTDYFDY (SEQ ID NO: 48);
a complementarity-determining region (CDR) light chain 1 (CDR-L1) including the amino acid sequence of QSISSY (SEQ ID NO: 4), QTIGSY (SEQ ID NO: 21), SGSIASNY (SEQ ID NO: 31), QGINDF (SEQ ID NO: 40), QSVSSSY (SEQ ID NO: 49);
a complementarity-determining region (CDR) light chain 2 (CDR-L2) including the amino acid sequence of AAS (SEQ ID NO: 5), GAS (SEQ ID NO: 22), or EDK (SEQ ID NO: 32); and
a complementarity-determining region (CDR) light chain 3 (CDR-L3) including the amino acid sequence of QQTYSTPLT (SEQ ID NO: 6), QQSYSTPFT (SEQ ID NO: 11), QQSYSTPLT (SEQ ID NO: 17), QSYDSSNHWV (SEQ ID NO: 33), or QQDYNSPYT (SEQ ID NO: 50).

In some embodiments, the antibody or antigen-binding fragment includes a CDR-H1 including the amino acid sequence of GFTFSSYA (SEQ ID NO: 1).

In some embodiments, the antibody or antigen-binding fragment includes a CDR-H2 including the amino acid sequence of ISGSGGST (SEQ ID NO: 2).

In some embodiments, the antibody or antigen-binding fragment includes a CDR-L1 including the amino acid sequence of QSISSY (SEQ ID NO: 4).

In some embodiments, the antibody or antigen-binding fragment includes a CDR-L2 including the amino acid sequence of AAS (SEQ ID NO: 5).

In some embodiments, the antibody or antigen-binding fragment includes a CDR-L3 including the amino acid sequence of QQSYSTPFT (SEQ ID NO: 11).

In some embodiments, the antibody or antigen-binding fragment thereof includes: a CDR-H1 including the amino acid sequence of GFTFSSYA (SEQ ID NO: 1); a CDR-H2 including the amino acid sequence of ISGSGGST (SEQ ID NO: 2); a CDR-H3 including the amino acid sequence of AKDLDWSGHDAFDI (SEQ ID NO: 3); a CDR-L1 including the amino acid sequence of QSISSY (SEQ ID NO: 4); a CDR-L2 including the amino acid sequence of AAS (SEQ ID NO: 5); and a CDR-L3 including the amino acid sequence of QQTYSTPLT (SEQ ID NO: 6). In some embodiments, the antibody or antigen-binding fragment thereof includes: a heavy chain variable domain including an amino acid sequence with at least 90% (e.g., at least 95%, 96%, 97%, 98%, or 99%) sequence identity to the amino acid sequence of SEQ ID NO: 7; and a light chain variable domain including an amino acid sequence with at least 90% (e.g., at least 95%, 96%, 97%, 98%, or 99%) sequence identity to the amino acid sequence of SEQ ID NO: 8. In some embodiments, the antibody or antigen-binding fragment thereof includes: a heavy chain variable domain including the amino acid sequence of SEQ ID NO: 7; and a light chain variable domain including the amino acid sequence of SEQ ID NO: 8.

In some embodiments, the antibody or antigen-binding fragment thereof includes: a CDR-H1 including the amino acid sequence of GFTFSNAW (SEQ ID NO: 9); a CDR-H2 including the amino acid sequence of ISGSGGST (SEQ ID NO: 2); a CDR-H3 including the amino acid sequence of ARDRGYSYSETSNDAFDI (SEQ ID NO: 10); a CDR-L1 including the amino acid sequence of QSISSY (SEQ ID NO: 4); a CDR-L2 including the amino acid sequence of AAS (SEQ ID NO: 5); and a CDR-L3 including the amino acid sequence of QQSYSTPFT (SEQ ID NO: 11). In some embodiments, the antibody or antigen-binding fragment thereof includes: a heavy chain variable domain including an amino acid sequence with at least 90% (e.g., at least 95%, 96%, 97%, 98%, or 99%) sequence identity to the amino acid sequence of SEQ ID NO: 12; and a light chain variable domain including an amino acid sequence with at least 90% (e.g., at least 95%, 96%, 97%, 98%, or 99%) sequence identity to the amino acid sequence of SEQ ID NO: 13. In some embodiments, the antibody or antigen-binding fragment thereof includes: a heavy chain variable domain including the amino acid sequence of SEQ ID NO: 12;
and a light chain variable domain including the amino acid sequence of SEQ ID NO: 13.

In some embodiments, the antibody or antigen-binding fragment thereof includes: a CDR-H1 including the amino acid sequence of GFTFSSYS (SEQ ID NO: 14); a CDR-H2 including the amino acid sequence of ISSSSSTI (SEQ ID NO: 15); a CDR-H3 including the amino acid sequence of ARGPDLSDYFDY (SEQ ID NO: 16); a CDR-L1 including the amino acid sequence of QSISSY (SEQ ID NO: 4); a CDR-L2 including the amino acid sequence of AAS (SEQ ID NO: 5); and a CDR-L3 including the amino acid sequence of QQSYSTPLT (SEQ ID NO: 17). In some embodiments, the antibody or antigen-binding fragment thereof includes: a heavy chain variable domain including an amino acid sequence with at least 90% (e.g., at least 95%, 96%, 97%, 98%, or 99%) sequence identity to the amino acid sequence of SEQ ID NO: 12; and a light chain variable domain including an amino acid sequence with at least 90% (e.g., at least 95%, 96%, 97%, 98%, or 99%) sequence identity to the amino acid sequence of SEQ ID NO: 13. In some embodiments, the antibody or antigen-binding fragment thereof includes: a heavy chain variable domain including the amino acid sequence of SEQ ID NO: 12; and a light chain variable domain including the amino acid sequence of SEQ ID NO: 13.

In some embodiments, the antibody or antigen-binding fragment thereof includes: a CDR-H1 including the amino acid sequence of GFTFSSYA (SEQ ID NO: 1); a CDR-H2 including the amino acid sequence of ISGSGGST (SEQ ID NO: 2); a CDR-H3 including the amino acid sequence of AKDPRGSGRDDAFDI (SEQ ID NO: 20); a CDR-L1 including the amino acid sequence of QTIGSY (SEQ ID NO: 21); a CDR-L2 including the amino acid sequence of GAS (SEQ ID NO: 22); and a CDR-L3 including the amino acid sequence of QQSYSTPFT (SEQ ID NO: 11). In some embodiments, the antibody or antigen-binding fragment thereof includes: a heavy chain variable domain including an amino acid sequence with at least 90% (e.g., at least 95%, 96%, 97%, 98%, or 99%) sequence identity to the amino acid sequence of SEQ ID NO: 23; and a light chain variable domain including an amino acid sequence with at least 90% (e.g., at least 95%, 96%, 97%, 98%, or 99%) sequence identity to the amino acid sequence of SEQ ID NO: 24. In some embodiments, the antibody or antigen-binding fragment thereof includes: a heavy chain variable domain including the amino acid sequence of SEQ ID NO: 23; and a light chain variable domain including the amino acid sequence of SEQ ID NO: 24.

In some embodiments, the antibody or antigen-binding fragment thereof includes: a CDR-H1 including the amino acid sequence of GFTFSSYA (SEQ ID NO: 1); a CDR-H2 including the amino acid sequence of ISGSGGST (SEQ ID NO: 2); a CDR-H3 including the amino acid sequence of AKDPTTMTTDAFDI (SEQ ID NO: 25); a CDR-L1 including the amino acid sequence of QSISSY (SEQ ID NO: 4); a CDR-L2 including the amino acid sequence of AAS (SEQ ID NO: 5); and a CDR-L3 including the amino acid sequence of QQSYSTPFT (SEQ ID NO: 11). In some embodiments, the antibody or antigen-binding fragment thereof includes: a heavy chain variable domain including an amino acid sequence with at least 90% (e.g., at least 95%, 96%, 97%, 98%, or 99%) sequence identity to the amino acid sequence of SEQ ID NO: 26; and a light chain variable domain including an amino acid sequence with at least 90% (e.g., at least 95%, 96%, 97%, 98%, or 99%) sequence identity to the amino acid sequence of SEQ ID NO: 27. In some embodiments, the antibody or antigen-binding fragment thereof includes: a heavy chain variable domain including the amino acid sequence of SEQ ID NO: 26; and a light chain variable domain including the amino acid sequence of SEQ ID NO: 27.

In some embodiments, the antibody or antigen-binding fragment thereof includes: a CDR-H1 including the amino acid sequence of GYTFTDYY (SEQ ID NO: 28); a CDR-H2 including the amino acid sequence of FDPEDGET (SEQ ID NO: 29); a CDR-H3 including the amino acid sequence of ATLDYRGVVYFDY (SEQ ID NO: 30); a CDR-L1 including the amino acid sequence of SGSIASNY (SEQ ID NO: 31); a CDR-L2 including the amino acid sequence of EDK (SEQ ID NO: 32); and a CDR-L3 including the amino acid sequence of QSYDSSNHWV (SEQ ID NO: 33). In some embodiments, the antibody or antigen-binding fragment thereof includes: a heavy chain variable domain including an amino acid sequence with at least 90% (e.g., at least 95%, 96%, 97%, 98%, or 99%) sequence identity to the amino acid sequence of SEQ ID NO: 34; and a light chain variable domain including an amino acid sequence with at least 90% (e.g., at least 95%, 96%, 97%, 98%, or 99%) sequence identity to the amino acid sequence of SEQ ID NO: 35. In some embodiments, the antibody or antigen-binding fragment thereof includes: a heavy chain variable domain including the amino acid sequence of SEQ ID NO: 34;
and a light chain variable domain including the amino acid sequence of SEQ ID NO: 35.

In some embodiments, the antibody or antigen-binding fragment thereof includes: a CDR-H1 including the amino acid sequence of GFTFSDYW (SEQ ID NO: 36); a CDR-H2 including the amino acid sequence of ISGSGGST (SEQ ID NO: 2); a CDR-H3 including the amino acid sequence of AKDLLWAARDAFDI (SEQ ID NO: 37); a CDR-L1 including the amino acid sequence of QSISSY (SEQ ID NO: 4); a CDR-L2 including the amino acid sequence of AAS (SEQ ID NO: 5); and a CDR-L3 including the amino acid sequence of QQSYSTPFT (SEQ ID NO: 11). In some embodiments, the antibody or antigen-binding fragment thereof includes: a heavy chain variable domain including an amino acid sequence with at least 90% (e.g., at least 95%, 96%, 97%, 98%, or 99%) sequence identity to the amino acid sequence of SEQ ID NO: 38; and a light chain variable domain including an amino acid sequence with at least 90% (e.g., at least 95%, 96%, 97%, 98%, or 99%) sequence identity to the amino acid sequence of SEQ ID NO: 39. In some embodiments, the antibody or antigen-binding fragment thereof includes: a heavy chain variable domain including the amino acid sequence of SEQ ID NO: 38; and a light chain variable domain including the amino acid sequence of SEQ ID NO: 39.

In some embodiments, the antibody or antigen-binding fragment thereof includes: a CDR-H1 including the amino acid sequence of GFTFSSYA (SEQ ID NO: 1); a CDR-H2 including the amino acid sequence of ISGSGGST (SEQ ID NO: 2); a CDR-H3 including the amino acid sequence of AKDLLWAARDAFDI (SEQ ID NO: 37); a CDR-L1 including the amino acid sequence of QGINDF (SEQ ID NO: 40); a CDR-L2 including the amino acid sequence of AAS (SEQ ID NO: 5); and a CDR-L3 including the amino acid sequence of QQSYSTPLT (SEQ ID NO: 17). In some embodiments, the antibody or antigen-binding fragment thereof includes: a heavy chain variable domain including an amino acid sequence with at least 90% (e.g., at least 95%, 96%, 97%, 98%, or 99%) sequence identity to the amino acid sequence of SEQ ID NO: 41; and a light chain variable domain including an amino acid sequence with at least 90% (e.g., at least 95%, 96%, 97%, 98%, or 99%) sequence identity to the amino acid sequence of SEQ ID NO: 42. In some embodiments, the antibody or antigen-binding fragment thereof includes: a heavy chain variable domain including the amino acid sequence of SEQ ID NO: 41; and a light chain variable domain including the amino acid sequence of SEQ ID NO: 42.

In some embodiments, the antibody or antigen-binding fragment thereof includes: a CDR-H1 including the amino acid sequence of GFTFSNAW (SEQ ID NO: 9); a CDR-H2 including the amino acid sequence of ISGSGGST (SEQ ID NO: 2); a CDR-H3 including the amino acid sequence of AKQTVTSADDYFDY (SEQ ID NO: 43); a CDR-L1 including the amino acid sequence of QSISSY (SEQ ID NO: 4); a CDR-L2 including the amino acid sequence of AAS (SEQ ID NO: 5); and a CDR-L3 including the amino acid sequence of QQSYSTPFT (SEQ ID NO: 11). In some embodiments, the antibody or antigen-binding fragment thereof includes: a heavy chain variable domain including an amino acid sequence with at least 90% (e.g., at least 95%, 96%, 97%, 98%, or 99%) sequence identity to the amino acid sequence of SEQ ID NO: 44; and a light chain variable domain including an amino acid sequence with at least 90% (e.g., at least 95%, 96%, 97%, 98%, or 99%) sequence identity to the amino acid sequence of SEQ ID NO: 45. In some embodiments, the antibody or antigen-binding fragment thereof includes: a heavy chain variable domain including the amino acid sequence of SEQ ID NO: 44; and a light chain variable domain including the amino acid sequence of SEQ ID NO: 45.

In some embodiments, the antibody or antigen-binding fragment thereof includes: a CDR-H1 including the amino acid sequence of GFMFDTHA (SEQ ID NO: 46); a CDR-H2 including the amino acid sequence of ISGSGGSI (SEQ ID NO: 47); a CDR-H3 including the amino acid sequence of ARSGETAGTDYFDY (SEQ ID NO: 48); a CDR-L1 including the amino acid sequence of QSVSSSY (SEQ ID NO: 49); a CDR-L2 including the amino acid sequence of GAS (SEQ ID NO: 22); and a CDR-L3 including the amino acid sequence of QQDYNSPYT (SEQ ID NO: 50). In some embodiments, the antibody or antigen-binding fragment thereof includes: a heavy chain variable domain including an amino acid sequence with at least 90% (e.g., at least 95%, 96%, 97%, 98%, or 99%) sequence identity to the amino acid sequence of SEQ ID NO: 51; and a light chain variable domain including an amino acid sequence with at least 90% (e.g., at least 95%, 96%, 97%, 98%, or 99%) sequence identity to the amino acid sequence of SEQ ID NO: 52. In some embodiments, the antibody or antigen-binding fragment thereof includes: a heavy chain variable domain including the amino acid sequence of SEQ ID NO: 51; and a light chain variable domain including the amino acid sequence of SEQ ID NO: 52.

In some embodiments, the antibody or antigen-binding fragment thereof includes a heavy chain variable domain including an amino acid sequence with at least 90% (e.g., at least 95%, 96%, 97%, 98%, 99%, or 100%) sequence identity to the amino acid sequence of any one of SEQ ID NOs: 7, 12, 18, 23, 26, 34, 38, 41, 44, or 51.

In some embodiments, the antibody or antigen-binding fragment thereof includes a light chain variable domain including an amino acid sequence with at least 90% (e.g., at least 95%, 96%, 97%, 98%, 99%, or 100%) sequence identity to the amino acid sequence of any one of SEQ ID NOs: 8, 13, 19, 24, 35, 39, 42, 45, or 52.

In some embodiments, the antibody or antigen-binding fragment thereof is selected from the group consisting of a monoclonal antibody or antigen-binding fragment thereof, a polyclonal antibody or antigen-binding fragment thereof, a human antibody or antigen-binding fragment thereof, a humanized antibody or antigen-binding fragment thereof, a primatized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, a nanobody, an antibody-like protein scaffold, a domain antibody, a Fv fragment, a Fab fragment, a F(ab′)₂ molecule, and a tandem scFv (taFv).

In some embodiments, the antibody or antigen-binding fragment thereof is a human, humanized, or chimeric antibody or antigen-binding fragment thereof.

In some embodiments, the antibody or antigen-binding fragment thereof binds specifically to a heterodimer of ITGA11 and integrin β1 (ITGA11β1).

In another aspect, the disclosure provides a polynucleotide encoding any antibody or antigen-binding fragment thereof described herein.

In another aspect, the disclosure provides a vector including a polynucleotide described herein (e.g., a polynucleotide encoding any antibody or antigen-binding fragment thereof described herein). In some embodiments, the vector is an expression vector (e.g., eukaryotic expression vector or a viral vector, such as a viral vector selected from the group consisting of adenovirus (Ad), retrovirus, poxvirus, adeno-associated virus, baculovirus, herpes simplex virus, and a vaccinia virus).

In another aspect, the disclosure provides a host cell including a vector described herein. In some embodiments, the host cell expresses an antibody or antigen-binding fragment thereof described herein.

In another aspect, the disclosure provides a pharmaceutical composition including an antibody or antigen-binding fragment thereof described herein, a polynucleotide described herein, a vector described herein, or a host cell described herein, and a pharmaceutically acceptable carrier or excipient.

In another aspect, the disclosure provides a kit including an agent selected from an antibody or antigen-binding fragment thereof described herein, a polynucleotide described herein, a vector described herein, or a host cell described herein, or a pharmaceutical composition described herein.

In another aspect, the disclosure provides a method of treating a subject having or at risk of developing a disorder, the method including administering to the subject an antibody or antigen-binding fragment thereof described herein, a polynucleotide described herein, a vector described herein, or a host cell described herein, or a pharmaceutical composition described herein.

In some embodiments, the disorder is a fibrotic disorder. In some embodiments, the fibrotic disorder is selected from hepatic fibrosis (e.g., fibrosis associated with cirrhosis (e.g., alcohol-induced cirrhosis, viral-induced cirrhosis, post-hepatitis C cirrhosis, and primary biliary cirrhosis), schistosomiasis, cholangitis (e.g., sclerosing cholangitis), and autoimmune-induced hepatitis), kidney fibrosis (e.g., tubulointerstitial fibrosis, scleroderma, diabetic nephritis, and glomerular nephritis), dermal fibrosis (e.g., scleroderma, hypertrophic and keloid scarring, nephrogenic fibrosing dermatopathy, and burns), myelofibrosis, neurofibromatosis, fibroma, intestinal fibrosis, and fibrotic adhesions resulting from surgical procedures), heart fibrosis (e.g., fibrosis associated with myocardial infarction), vascular fibrosis (e.g., fibrosis associated with postangioplasty arterial restenosis and atherosclerosis), ocular fibrosis (e.g., fibrosis associated with post-cataract surgery, proliferative vitreoretinopathy, and retro-orbital fibrosis), bone marrow fibrosis (e.g., idiopathic myelofibrosis and drug-induced myelofibrosis), pulmonary fibrosis (e.g., pulmonary interstitial fibrosis), glomerulonephritis, heart failure (ischemic and non-ischemic), scleroderma, excessive scar tissue post-surgery or device insertion, trauma or burns, progressive kidney disease, valvular heart disease, hypertensive heart disease, articular and periarticular fibrosis, myelofibrosis, ocular/vitreous fibrosis, intestinal fibrosis and stricture, peritoneal and retro-peritoneal fibrosis, pancreatic fibrosis, nephrogenic systemic fibrosis, primary sclerosing cholangitis, and the elaboration of pathological matrix also has a role in fibroproliferative tumor progression and metastasis. In some embodiments, the disorder is an inflammatory disorder. In some embodiments, the inflammatory disorder is selected from asthma (e.g., allergic asthma, exercise-induced asthma, aspirin sensitive/exacerbated asthma, atopic asthma, severe asthma, mild asthma, moderate to severe asthma, corticosteroid naïve asthma, chronic asthma, corticosteroid resistant asthma, corticosteroid refractory asthma, newly diagnosed and untreated asthma, asthma due to smoking, asthma uncontrolled on corticosteroids, and the like), airway inflammation, airway hyperreactivity, airway hyperresponsiveness, rhinosinusitis, rhinosinusitis with polyps, nasal polyposis, arthritis (e.g., osteoarthritis, rheumatoid arthritis, collagen-induced arthritis, arthritic joints as a result of injury, etc.), eosinophilic inflammation, mast cell-mediated inflammatory diseases, sepsis, septic shock, seronegative enthesopathy and arthropathy (SEA) syndrome, osteoporosis, eosinophilic esophagitis, scleroderma, dermatitis, atopic dermatitis, allergic rhinitis, bullous pemphigoid, urticaria (e.g., chronic urticaria), cartilage inflammation, polymyalgia rheumatic, polyarteritis nodossa, Wegener's granulomatosis, Behcet's disease, myolitis, polymyolitis, dermatomyolitis, dermatomyositis, vasculitis, arteritis, diabetic nephropathy, interstitial cystitis, graft versus host disease (GVHD), gastrointestinal inflammatory conditions (e.g., inflammatory bowel disease (IBD), ulcerative colitis (UC), Crohn's disease (CD), colitis (e.g., colitis caused by environmental insults (e.g., caused by or associated with a therapeutic regimen, such as chemotherapy, radiation therapy, and the like), infectious colitis, ischemic colitis, collagenous or lymphocytic colitis, necrotizing enterocolitis, colitis in conditions such as chronic granulomatous disease or celiac disease, food allergies, gastritis, infectious gastritis or enterocolitis (e.g., Helicobacter pylori-infected chronic active gastritis), and other forms of gastrointestinal inflammation caused by an infectious agent), and inflammatory pulmonary conditions (e.g., chronic obstructive pulmonary disease (COPD), eosinophilic pulmonary inflammation, infection-induced pulmonary conditions (including those associated with viral (e.g., influenza, parainfluenza, rotavirus, human metapneumovirus, and respiratory syncytial virus), bacterial, fungal (e.g., Aspergillus), parasitic, or prion infection, allergen-induced pulmonary conditions, pollutant-induced pulmonary conditions (e.g., asbestosis, silicosis, or berylliosis), gastric aspiration-induced pulmonary conditions, immune dysregulation, inflammatory conditions with genetic predisposition such as cystic fibrosis, physical trauma-induced pulmonary conditions (e.g., ventilator injury), emphysema, bronchitis, sarcoidosis, histiocytosis, lymphangiomyomatosis, acute lung injury, acute respiratory distress syndrome, chronic lung disease, bronchopulmonary dysplasia, pneumonia (e.g., community-acquired pneumonia, nosocomial pneumonia, ventilator-associated pneumonia, viral pneumonia, bacterial pneumonia, and severe pneumonia), airway exacerbations, and acute respiratory distress syndrome (ARDS)).

In some embodiments, the disorder is a cancer. In some embodiments, the cancer is selected from breast cancer, colorectal cancer, hepatic cancer, kidney cancer, liver cancer, lung cancer, pancreatic cancer, gastrointestinal cancer, melanoma, ovarian cancer, prostate cancer, cervical cancer, bladder cancer, glioblastoma, head and neck cancer, and cholangiocarcinoma.

### Definitions

To facilitate the understanding of this disclosure, a number of terms are defined below. Terms defined herein have meanings as commonly understood by a person of ordinary skill in the areas relevant to the disclosure. Terms such as “a”, “an,” and “the” are not intended to refer to only a singular entity, but include the general class of which a specific example may be used for illustration. The terminology herein is used to describe specific embodiments of the disclosure, but their usage does not limit the disclosure, except as outlined in the claims.

As used herein, the term “about” refers to a value that is no more than 10% above or below the value being described. For example, the term “about 5 nM” indicates a range of from 4.5 nM to 5.5 nM.

As used herein, any values provided in a range of values include both the upper and lower bounds and any values contained within the upper and lower bounds.

As used herein, the term “antibody” (Ab) refers to an immunoglobulin molecule that specifically binds to, or is immunologically reactive with, a particular antigen, and includes polyclonal, monoclonal, genetically engineered and otherwise modified forms of antibodies, including but not limited to chimeric antibodies, humanized antibodies, primatized antibodies, heteroconjugate antibodies (e.g., bi-tri- and quad-specific antibodies, diabodies, triabodies, and tetrabodies), and antigen-binding fragments of antibodies, including e.g., Fab′, F(ab′)₂, Fab, Fv, rIgG, and scFv fragments. Moreover, unless otherwise indicated, the term “monoclonal antibody” (mAb) is meant to include both intact molecules, as well as antibody fragments (such as, for example, Fab and F(ab′)₂ fragments) that are capable of specifically binding to a target protein. Fab and F(ab′)₂ fragments lack the Fc fragment of an intact antibody, clear more rapidly from the circulation of the animal, and may have less non-specific tissue binding than an intact antibody (see Wahl et al., J. Nucl. Med. 24:316, 1983; incorporated herein by reference).

The term “antigen-binding fragment,” as used herein, refers to one or more fragments of an antibody that retain the ability to specifically bind to a target antigen (e.g., as measured by binding affinity). The antigen-binding function of an antibody can be performed by fragments of a full-length antibody. The antibody fragments can be a Fab, F(ab′)₂, scFv, SMIP, diabody, a triabody, an affibody, a nanobody, an aptamer, a bispecific, a dual-binding bispecific, a mAb pair or a domain antibody. Examples of binding fragments encompassed of the term “antigen-binding fragment” of an antibody include, but are not limited to: (i) a Fab fragment, a monovalent fragment consisting of the VL, VH, CL, and CH1 domains; (ii) a F(ab′)₂ fragment, a bivalent fragment including two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb including VH and VL domains; (vi) a dAb fragment (Ward et al., Nature 341:544-546, 1989), which consists of a VH domain; (vii) a dAb which consists of a VH or a VL domain; (viii) an isolated complementarity determining region (CDR); and (ix) a combination of two or more isolated CDRs which may optionally be joined by a synthetic linker. Furthermore, although the two domains of the Fv fragment, VL and VH, are coded for by separate genes, they can be joined, using recombinant methods, by a linker that enables them to be made as a single protein chain in which the VL and VH regions pair to form monovalent molecules (known as single-chain Fv (scFv); see, e.g., Bird et al., Science 242:423-426, 1988, and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in some embodiments, by chemical peptide synthesis procedures known in the art.

As used herein, the term “binding affinity” is meant the strength of the total noncovalent interactions between a single binding site of a molecule (e.g., an antibody) and its binding partner (e.g., an antigen or antigenic peptide). Unless otherwise indicated, as used herein, “binding affinity” refers to intrinsic binding affinity, which reflects a specific interaction between members of a binding pair (e.g., antibody and antigen). The affinity of a molecule X for its partner Y can generally be represented by the dissociation constant (Kd). Affinity can be measured by standard methods known in the art, including those described herein. A low-affinity complex contains an antibody that generally tends to dissociate readily from the antigen, whereas a high-affinity complex contains an antibody that generally tends to remain bound to the antigen for a longer duration.

As used herein, the term “chimeric” antibody refers to an antibody having variable domain sequences (e.g., CDR sequences) derived from an immunoglobulin of one source organism, such as rat or mouse, and constant regions derived from an immunoglobulin of a different organism (e.g., a human, another primate, pig, goat, rabbit, hamster, cat, dog, guinea pig, member of the bovidae family (such as cattle, bison, buffalo, elk, and yaks, among others), cow, sheep, horse, or bison, among others). Methods for producing chimeric antibodies are known in the art. See, e.g., Morrison, 1985, Science 229 (4719): 1202-7; Oi et al, 1986, BioTechniques 4:214-221; Gillies et al, 1985, J. Immunol. Methods 125:191-202; U.S. Pat. Nos. 5,807,715; 4,816,567; and 4,816,397; incorporated herein by reference.

As used herein, the term “complementarity determining region” (CDR) refers to a hypervariable region found both in the light chain and the heavy chain variable domains. The more highly conserved portions of variable domains are called the framework regions (FRs). As is appreciated in the art, the amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The antibodies described herein may including modifications in these hybrid hypervariable positions. The variable domains of native heavy and light chains each include four framework regions that primarily adopt a β-sheet configuration, connected by three CDRs, which form loops that connect, and in some cases form part of, the β-sheet structure. The CDRs in each chain are held together in close proximity by the FR regions in the order FR1-CDR1-FR2-CDR2-FR3-CDR3-FR4 and, with the CDRs from the other antibody chains, contribute to the formation of the target binding site of antibodies (see Kabat et al, Sequences of Proteins of Immunological Interest (National Institute of Health, Bethesda, Md. 1987; incorporated herein by reference). As used herein, numbering of immunoglobulin amino acid residues is done according to the immunoglobulin amino acid residue numbering system of Kabat et al, unless otherwise indicated.

As used herein, the terms “conservative mutation,” “conservative substitution,” or “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in table 1 below.

TABLE 1
Representative physicochemical properties
of naturally-occurring amino acids

| | | | | Electrostatic | |
|---|---|---|---|---|---|
| | 3 | 1 | Side- | character at | |
| | Letter | Letter | chain | physiological pH | Steric |
| Amino Acid | Code | Code | Polarity | (7.4) | Volume^{†} |
| Alanine | Ala | A | nonpolar | Neutral | small |
| Arginine | Arg | R | polar | Cationic | large |
| Asparagine | Asn | N | polar | Neutral | intermediate |
| Aspartic acid | Asp | D | polar | Anionic | intermediate |
| Cysteine | Cys | C | nonpolar | Neutral | intermediate |
| Glutamic | Glu | E | polar | Anionic | intermediate |
| acid | | | | | |
| Glutamine | Gln | Q | polar | Neutral | intermediate |
| Glycine | Gly | G | nonpolar | Neutral | small |
| Histidine | His | H | polar | Both neutral and | large |
| | | | | cationic forms in | |
| | | | | equilibrium at | |
| | | | | pH 7.4 | |
| Isoleucine | Ile | I | nonpolar | Neutral | large |
| Leucine | Leu | L | nonpolar | Neutral | large |
| Lysine | Lys | K | polar | Cationic | large |
| Methionine | Met | M | nonpolar | Neutral | large |
| Phenyl- | Phe | F | nonpolar | neutral | large |
| alanine | | | | | |
| Proline | Pro | P | non- | neutral | intermediate |
| | | | polar | | |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include, e.g., (i) G, A, V, L, I, P, and M; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

Amino acid substitutions may be represented herein using the convention: (AA1) (N)(AA2), where “AA1” represents the amino acid normally present at particular site within an amino acid sequence, “N” represents the residue number within the amino acid sequence at which the substitution occurs, and “AA2” represents the amino acid present in the amino acid sequence after the substitution is effectuated. For example, the notation “C232S” in the context of an antibody hinge region, such as an IgG2 antibody hinge region, refers to a substitution of the naturally-occurring cysteine residue for a serine residue at amino acid residue 232 of the indicated hinge amino acid sequence. Likewise, the notation “C233S” in the context of an antibody hinge region, such as an IgG2 antibody hinge region, refers to a substitution of the naturally-occurring cysteine residue for a serine residue at amino acid residue 233 of the indicated hinge amino acid sequence.

As used herein, the term “conjugate” refers to a compound formed by the chemical bonding of a reactive functional group of one molecule with an appropriately reactive functional group of another molecule.

As used herein, the term “derivatized antibodies” refers to antibodies that are modified by a chemical reaction so as to cleave residues or add chemical moieties not native to an isolated antibody. Derivatized antibodies can be obtained by glycosylation, acetylation, pegylation, phosphorylation, amidation, derivatization by addition of known chemical protecting/blocking groups, proteolytic cleavage, linkage to a cellular ligand or other protein. Any of a variety of chemical modifications can be carried out by known techniques, including, without limitation, specific chemical cleavage, acetylation, formylation, metabolic synthesis of tunicamycin, etc. using established procedures. Additionally, the derivative can contain one or more non-natural amino acids, e.g., using amber suppression technology (see, e.g., U.S. Pat. No. 6,964,859; incorporated herein by reference).

As used herein, the term “diabodies” refers to bivalent antibodies including two polypeptide chains, in which each polypeptide chain includes VH and VL domains joined by a linker that is too short (e.g., a linker composed of five amino acids) to allow for intramolecular association of VH and VL domains on the same peptide chain. This configuration forces each domain to pair with a complementary domain on another polypeptide chain so as to form a homodimeric structure. Accordingly, the term “triabodies” refers to trivalent antibodies including three peptide chains, each of which contains one VH domain and one VL domain joined by a linker that is exceedingly short (e.g., a linker composed of 1-2 amino acids) to permit intramolecular association of VH and VL domains within the same peptide chain. In order to fold into their native structure, peptides configured in this way typically trimerize so as to position the VH and VL domains of neighboring peptide chains spatially proximal to one another to permit proper folding (see Holliger et al., Proc. Natl. Acad. Sci. USA 90:6444-48, 1993; incorporated herein by reference).

As used herein, the term “disorder” refers to any condition, disease, or state of pathogenic abnormal biological function in a subject. In particular, the disclosure provides disorders associated ITGA11. Particular disorders of the disclosure include fibrotic disorders, inflammatory disorders, or cancers.

As used herein, the term “epitope” refers to a portion of an antigen that is recognized and bound by a polypeptide, such as an antibody, antigen-binding fragment thereof, single-chain polypeptide, or construct as described herein. In the context of a protein antigen (such as ITGA11), an epitope may be a continuous epitope, which is a single, uninterrupted segment of one or more amino acids covalently linked to one another by peptide bonds in which all of the component amino acids bind the polypeptide (e.g., antibody, antigen-binding fragment thereof, single-chain polypeptide, or construct thereof). Continuous epitopes may be composed, for instance, of 1, 5, 10, 15, 20, or more amino acids within an antigen. In some embodiments, an epitope may be a discontinuous epitope, which contains two or more segments of amino acids each separated from one another in an antigen's amino acid sequence by one or more intervening amino acid residues. Discontinuous epitopes may be composed, for instance, of 1, 2, 3, 4, 5, 6, 7, 8, 9, 10, or more such segments of amino acid residues. Despite this separation by intervening amino acids, the segments that compose a discontinuous epitope may be, for instance, spatially proximal to one another in the three-dimensional conformation of the antigen. An epitope may be defined not just by its amino acid compositions, but also by the post-translation state of an amino acid of the epitope (e.g., phosphorylation) or the bond geometry of a peptide bond between two amino acids in the epitope (e.g., cis or trans).

As used herein, the term “framework region” or “FW region” includes amino acid residues that are adjacent to the CDRs. FW region residues may be present in, for example, human antibodies, rodent-derived antibodies (e.g., murine antibodies), humanized antibodies, primatized antibodies, chimeric antibodies, antibody fragments (e.g., Fab fragments), single-chain antibody fragments (e.g., scFv fragments), antibody domains, and bispecific antibodies, among others.

As used herein, the term “fusion protein” refers to a protein that is joined via a covalent bond to another molecule. A fusion protein can be chemically synthesized by, e.g., an amide-bond forming reaction between the N-terminus of one protein to the C-terminus of another protein. Alternatively, a fusion protein containing one protein covalently bound to another protein can be expressed recombinantly in a cell (e.g., a eukaryotic cell or prokaryotic cell) by expression of a polynucleotide encoding the fusion protein, for example, from a vector or the genome of the cell. A fusion protein may contain one protein that is covalently bound to a linker, which in turn is covalently bound to another molecule. Examples of linkers that can be used for the formation of a fusion protein include peptide-containing linkers, such as those that contain naturally occurring or non-naturally occurring amino acids. In some embodiments, it may be desirable to include D-amino acids in the linker, as these residues are not present in naturally-occurring proteins and are thus more resistant to degradation by endogenous proteases. Linkers can be prepared using a variety of strategies that are well known in the art, and depending on the reactive components of the linker, can be cleaved by enzymatic hydrolysis, photolysis, hydrolysis under acidic conditions, hydrolysis under basic conditions, oxidation, disulfide reduction, nucleophilic cleavage, or organometallic cleavage (Leriche et al., Bioorg. Med. Chem., 20:571-582, 2012).

As used herein, the term “heterospecific antibodies” refers to monoclonal, preferably human or humanized, antibodies that have binding specificities for at least two different antigens. Traditionally, the recombinant production of heterospecific antibodies is based on the co-expression of two immunoglobulin heavy chain-light chain pairs, where the two heavy chains have different specificities (Milstein et al., Nature 305:537, 1983). Similar procedures are disclosed, e.g., in WO 93/08829, U.S. Pat. Nos. 6,210,668; 6,193,967; 6,132,992; 6,106,833; 6,060,285; 6,037,453; 6,010,902; 5,989,530; 5,959,084; 5,959,083; 5,932,448; 5,833,985; 5,821,333; 5,807,706; 5,643,759, 5,601,819; 5,582,996, 5,496,549, 4,676,980, WO 91/00360, WO 92/00373, EP 03089, Traunecker et al., EMBO J. 10:3655 (1991), Suresh et al., Methods in Enzymology 121:210 (1986); incorporated herein by reference. Heterospecific antibodies can include Fc mutations that enforce correct chain association in multi-specific antibodies, as described by Klein et al, mAbs 4 (6): 653-663, 2012; incorporated herein by reference.

As used herein, the term “human antibody” refers to an antibody in which substantially every part of the protein (e.g., CDR, framework, CL, CH domains (e.g., CH1, CH2, CH3), hinge, (VL, VH)) is substantially non-immunogenic in humans, with only minor sequence changes or variations. A human antibody can be produced in a human cell (e.g., by recombinant expression), or by a non-human animal or a prokaryotic or eukaryotic cell that is capable of expressing functionally rearranged human immunoglobulin (e.g., heavy chain and/or light chain) genes. Further, when a human antibody is a single-chain antibody, it can include a linker peptide that is not found in native human antibodies. For example, an Fv can include a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. See U.S. Pat. Nos. 4,444,887 and 4,716,111; and PCT publications WO 1998/46645; WO 1998/50433; WO 1998/24893; WO 1998/16654; WO 1996/34096; WO 1996/33735; and WO 1991/10741; incorporated herein by reference. Human antibodies can also be produced using transgenic mice that are incapable of expressing functional endogenous immunoglobulins, but which can express human immunoglobulin genes. See, e.g., PCT publications WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598; incorporated by reference herein.

As used herein, the term “humanized” antibody refers to forms of non-human (e.g., murine) antibodies that are chimeric immunoglobulins, immunoglobulin chains or fragments thereof (such as Fv, Fab, Fab′, F(ab′)₂ or other target-binding subdomains of antibodies) which contain minimal sequences derived from non-human immunoglobulin. In general, the humanized antibody will include substantially all of at least one, and typically two, variable domains, in which all or substantially all of the CDR regions correspond to those of a non-human immunoglobulin. All or substantially all of the FR regions may also be those of a human immunoglobulin sequence. The humanized antibody can also include at least a portion of an immunoglobulin constant region (Fc), typically that of a human immunoglobulin consensus sequence. Methods of antibody humanization are known in the art. See, e.g., Riechmann et al., Nature 332:323-7, 1988; U.S. Patent Nos: 5,530, 101; 5,585,089; 5,693,761; 5,693,762; and U.S. Pat. No. 6,180,370 to Queen et al; EP239400; PCT publication WO 91/09967; U.S. Pat. No. 5,225,539; EP592106; and EP519596; incorporated herein by reference.

As used herein, the term “hydrophobic side-chain” refers to an amino acid side-chain that exhibits low solubility in water relative due to, e.g., the steric or electronic properties of the chemical moieties present within the side-chain. Examples of amino acids containing hydrophobic side-chains include those containing unsaturated aliphatic hydrocarbons, such as alanine, valine, leucine, isoleucine, proline, and methionine, as well as amino acids containing aromatic ring systems that are electrostatically neutral at physiological pH, such as tryptophan, phenylalanine, and tyrosine.

As used herein, the term “monoclonal antibody” refers to an antibody that is derived from a single clone, including any eukaryotic, prokaryotic, or phage clone, and not the method by which it is produced. As used herein, the term “non-native constant region” refers to an antibody constant region that is derived from a source that is different from the antibody variable region or that is a human-generated synthetic polypeptide having an amino sequence that is different from the native antibody constant region sequence. For instance, an antibody containing a non-native constant region may have a variable region derived from a non-human source (e.g., a mouse, rat, or rabbit) and a constant region derived from a human source (e.g., a human antibody constant region), or a constant region derived from another primate, pig, goat, rabbit, hamster, cat, dog, guinea pig, member of the bovidae family (such as cattle, bison, buffalo, elk, and yaks, among others), cow, sheep, horse, or bison, among others). As used herein, the term “percent (%) sequence identity” refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent sequence identity (e.g., gaps can be introduced in one or both of the candidate and reference sequences for optimal alignment and non-homologous sequences can be disregarded for comparison purposes). Alignment for purposes of determining percent sequence identity can be achieved in various ways that are within the skill in the art, for instance, using publicly available computer software, such as BLAST, ALIGN, or Megalign (DNASTAR) software. Those skilled in the art can determine appropriate parameters for measuring alignment, including any algorithms needed to achieve maximal alignment over the full length of the sequences being compared. For example, a reference sequence aligned for comparison with a candidate sequence may show that the candidate sequence exhibits from 50% to 100% sequence identity across the full length of the candidate sequence or a selected portion of contiguous amino acid (or nucleic acid) residues of the candidate sequence. The length of the candidate sequence aligned for comparison purposes may be, for example, at least 30%, (e.g., 30%, 40, 50%, 60%, 70%, 80%, 90%, or 100%) of the length of the reference sequence. When a position in the candidate sequence is occupied by the same amino acid residue as the corresponding position in the reference sequence, then the molecules are identical at that position.

As used herein, the term “primatized antibody” refers to an antibody including framework regions from primate-derived antibodies and other regions, such as CDRs and/or constant regions, from antibodies of a non-primate source. Methods for producing primatized antibodies are known in the art. See e.g., U.S. Pat. Nos. 5,658,570; 5,681,722; and 5,693,780; incorporated herein by reference. For instance, a primatized antibody or antigen-binding fragment thereof described herein can be produced by inserting the CDRs of a non-primate antibody or antigen-binding fragment thereof into an antibody or antigen-binding fragment thereof that contains one or more framework regions of a primate.

As used herein, the term “operatively linked” in the context of a polynucleotide fragment is intended to mean that the two polynucleotide fragments are joined such that the amino acid sequences encoded by the two polynucleotide fragments remain in-frame.

As used herein, the term “pharmacokinetic profile” refers to the absorption, distribution, metabolism, and clearance of a drug over time following administration of the drug to a patient.

As used herein, the term “regulatory sequence” includes promoters, enhancers and other expression control elements (e.g., polyadenylation signals) that control the transcription or translation of the antibody chain genes. Such regulatory sequences are described, for example, in Goeddel, Gene Expression Technology: Methods in Enzymology 185 (Academic Press, San Diego, CA, 1990); incorporated herein by reference.

As used herein, the term “scFv” refers to a single-chain Fv antibody in which the variable domains of the heavy chain and the light chain from an antibody have been joined to form one chain. scFv fragments contain a single polypeptide chain that includes the variable region of an antibody light chain (VL) (e.g., CDR-L1, CDR-L2, and/or CDR-L3) and the variable region of an antibody heavy chain (VH) (e.g., CDR-H1, CDR-H2, and/or CDR-H3) separated by a linker. The linker that joins the VL and VH regions of a scFv fragment can be a peptide linker composed of proteinogenic amino acids. Alternative linkers can be used to so as to increase the resistance of the scFv fragment to proteolytic degradation (e.g., linkers containing D-amino acids), in order to enhance the solubility of the scFv fragment (e.g., hydrophilic linkers such as polyethylene glycol-containing linkers or polypeptides containing repeating glycine and serine residues), to improve the biophysical stability of the molecule (e.g., a linker containing cysteine residues that form intramolecular or intermolecular disulfide bonds), or to attenuate the immunogenicity of the scFv fragment (e.g., linkers containing glycosylation sites). scFv molecules are known in the art and are described, e.g., in U.S. Pat. No. 5,892,019, Flo et al., (Gene 77:51, 1989); Bird et al., (Science 242:423, 1988); Pantoliano et al., (Biochemistry 30:10117, 1991); Milenic et al., (Cancer Research 51:6363, 1991); and Takkinen et al., (Protein Engineering 4:837, 1991). The VL and VH domains of a scFv molecule can be derived from one or more antibody molecules. It will also be understood by one of ordinary skill in the art that the variable regions of the scFv molecules described herein can be modified such that they vary in amino acid sequence from the antibody molecule from which they were derived. For example, in one embodiment, nucleotide or amino acid substitutions leading to conservative substitutions or changes at amino acid residues can be made (e.g., in CDR and/or framework residues). Alternatively, or in addition, mutations are made to CDR amino acid residues to optimize antigen binding using art recognized techniques. scFv fragments are described, for example, in WO 2011/084714; incorporated herein by reference.

As used herein, the phrase “specifically binds” refers to a binding reaction which is determinative of the presence of an antigen in a heterogeneous population of proteins and other biological molecules that is recognized, e.g., by an antibody or antigen-binding fragment thereof, with particularity. An antibody or antigen-binding fragment thereof that specifically binds to an antigen will bind to the antigen with a K_{D} of less than 100 nM. For example, an antibody or antigen-binding fragment thereof that specifically binds to an antigen will bind to the antigen with a K_{D} of up to 100 nM (e.g., between 1 pM and 100 nM). An antibody or antigen-binding fragment thereof that does not exhibit specific binding to a particular antigen or epitope thereof will exhibit a K_{D} of greater than 100 nM (e.g., greater than 500 nm, 1 μM, 100 UM, 500 uM, or 1 mM) for that particular antigen or epitope thereof. A variety of immunoassay formats may be used to select antibodies specifically immunoreactive with a particular protein or carbohydrate. For example, solid-phase ELISA immunoassays are routinely used to select antibodies specifically immunoreactive with a protein or carbohydrate. See, Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of immunoassay formats and conditions that can be used to determine specific immunoreactivity.

As used herein, the terms “subject” and “patient” refer to an organism that receives treatment for a particular disease or condition as described herein. Examples of subjects and patients include mammals, such as humans, primates, pigs, goats, rabbits, hamsters, cats, dogs, guinea pigs, members of the bovidae family (such as cattle, bison, buffalo, elk, and yaks, among others), cows, sheep, horses, and bison, among others, receiving treatment for diseases or conditions.

As used herein, the term “transfection” refers to any of a wide variety of techniques commonly used for the introduction of exogenous DNA into a prokaryotic or eukaryotic host cell, e.g., electroporation, lipofection, calcium-phosphate precipitation, DEAE-dextran transfection and the like. As used herein, the terms “treat” or “treatment” refer to therapeutic treatment, in which the object is to prevent or slow down (lessen) an undesired physiological change or disorder, such as the progression of a fibrotic disorder, inflammatory disorder, or cancer described herein. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Those in need of treatment include those already with the condition or disorder, as well as those prone to have the condition or disorder or those in which the condition or disorder is to be prevented.

As used herein the term “variable region CDR” includes amino acids in a CDR or complementarity determining region as identified using sequence or structure-based methods. As used herein, the term “CDR” or “complementarity determining region” refers to the noncontiguous antigen-binding sites found within the variable regions of both heavy and light chain polypeptides. These particular regions have been described by Kabat et al., J. Biol. Chem. 252:6609-6616, 1977 and Kabat, et al., Sequences of Proteins of Immunological Interest, Fifth Edition, U.S. Department of Health and Human Services, NIH Publication No. 91-3242, 1991; by Chothia et al., (J. Mol. Biol. 196:901-917, 1987), and by MacCallum et al., (J. Mol. Biol. 262:732-745, 1996) where the definitions include overlapping or subsets of amino acid residues when compared against each other. The term “CDR” may be, for example, a CDR as defined by Kabat based on sequence comparisons.

As used herein, the term “vector” includes a nucleic acid vector, e.g., a DNA vector, such as a plasmid, a RNA vector, virus or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/11026; incorporated herein by reference. Expression vectors described herein contain a polynucleotide sequence as well as, e.g., additional sequence elements used for the expression of proteins and/or the integration of these polynucleotide sequences into the genome of a mammalian cell. Certain vectors that can be used for the expression of antibodies and antibody fragments described herein include plasmids that contain regulatory sequences, such as promoter and enhancer regions, which direct gene transcription. Other useful vectors for expression of antibodies and antibody fragments contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “VH” refers to the variable region of an immunoglobulin heavy chain of an antibody, including the heavy chain of an Fv, scFv, or Fab. References to “VL” refer to the variable region of an immunoglobulin light chain, including the light chain of an Fv, scFv, dsFv or Fab. Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 Daltons, composed of two identical light (L) chains and two identical heavy (H) chains. Each heavy chain of a native antibody has at the amino terminus a variable domain (VH) followed by a number of constant domains. Each light chain of a native antibody has a variable domain at the amino terminus (VL) and a constant domain at the carboxy terminus.

### BRIEF DESCRIPTION OF THE DRAWINGS

FIG. 1 shows that anti-ITGA11 antibodies FIB-918-1, FIB-918-2, FIB-918-3 and FIB-918-4 inhibit binding of collagen to human ITGA11 expressed in mouse C2C12 cells.

FIG. 2 shows that anti-ITGA11 antibodies FIB-918-5, FIB-918-6, FIB-918-7 and FIB-918-8 inhibit binding of collagen to human ITGA11 expressed in mouse C2C12 in cells.

FIG. 3 shows that anti-ITGA11 antibodies FIB-918-9 and FIB-918-10 inhibit binding of collagen to human ITGA11 expressed in mouse C2C12 cells.

### DETAILED DESCRIPTION

The present disclosure provides antibodies or antigen-binding fragments thereof that bind specifically to integrin α11 (ITGA11). The disclosure also provides methods for treating an ITGA11-associated disorder in a subject, such as a fibrotic disorder, an inflammatory disorder, or a cancer. Also included in the disclosure are related pharmaceutical compositions, polynucleotides, vectors, host cells, methods of production, methods of treatment, diagnostic methods, and kits.

### I. Anti-ITGA11 Antibodies or Antigen-Binding Fragments Thereof

The disclosure provides antibodies or antigen-binding fragments thereof that bind specifically to integrin chain α11 (ITGA11). In some embodiments, an antibody or antigen-binding fragment thereof disclosed herein binds specifically to human ITGA11, cynomolgus ITGA11, and/or mouse ITGA11. In some embodiments, an antibody or antigen-binding fragment thereof disclosed herein binds specifically to ITGA11 heterodimerized with integrin chain β1 (ITGB1) (α11β1 or ITGA11β1) (e.g., human ITGA11β1, cynomolgus ITGA11β1, or mouse ITGA11β1). In some embodiments, an antibody or antigen-binding fragment thereof disclosed herein binds specifically to a human ITGA11 heterodimerized with human ITGB1, cynomolgus ITGB1, or mouse ITGB1.

In some embodiments, an antibody or antigen-binding fragment thereof disclosed herein binds specifically to ITGA11 (e.g., human ITGA11) over ITGA10 (e.g., human ITGA10) or ITGA2 (e.g. human ITGA2). In some embodiments, an antibody or antigen-binding fragment thereof disclosed herein binds specifically to ITGA11β1 (e.g., human ITGA11β1) over ITGA10β1 (e.g., human ITGA10β1) or ITGA2β1 (e.g., human ITGA2β1).

In some embodiments, an antibody or antigen-binding fragment thereof disclosed herein is a monovalent, bispecific antibody.

In some embodiments, an antibody or antigen-binding fragment thereof disclosed herein preferentially binds ITGA11 and ITGA10 compared to ITGA2 or ITGB1.

In particular, the disclosure features anti-ITGA11 antibodies or antigen-binding fragments described according to any of the CDR, heavy chain (VH), and/or light chain (VL) sequences provided below. The disclosure specifically contemplates antibodies having any combination of the disclosed CDRs (e.g., a combination of any CDR-H1, CDR-H2, CDR-H3, CDR-L1, CDR-L2, and CDR-L3 disclosed herein). The disclosure also specifically contemplates the pairing of a heavy chain including any heavy chain variable region (VH) described herein with the pairing of a light chain including any light chain variable region (VL) disclosed herein.

### mAb FIB-918-1

mAb FIB-918-1 includes a complementarity-determining region (CDR) heavy chain 1 (CDR-H1) including the amino acid sequence of GFTFSSYA (SEQ ID NO: 1); a CDR heavy chain 2 (CDR-H2) including the amino acid sequence of ISGSGGST (SEQ ID NO: 2); and a CDR heavy chain 3 (CDR-H3) including the amino acid sequence of AKDLDWSGHDAFDI (SEQ ID NO: 3).

mAb FIB-918-1 includes a heavy chain variable domain having an amino acid sequence of

EVQLVESGGGLVQPGGSLRLSCAASGFTFSSYAMSWVRQAPG
KGLEWVSAISGSGGSTYYADSVKGRFTISRDNSKNTLYLQMN
SLRAEDTAVYYCAKDLDWSGHDAFDIWGQGTTVTVSS
(SEQ ID NO: 7; CDRs underlined).

mAb FIB-918-1 includes a complementarity-determining region (CDR) light chain 1 (CDR-L1) including the amino acid sequence of QSISSY (SEQ ID NO: 4); a CDR light chain 2 (CDR-L2) including the amino acid sequence of AAS (SEQ ID NO: 5); and a CDR light chain 3 (CDR-L3) including the amino acid sequence of QQTYSTPLT (SEQ ID NO: 6).

mAb FIB-918-1 includes a light chain variable
domain having an amino acid sequence of
DIQMTQSPSSLSASVGDRVTITCRASQSISSYLNWYQQKPGKA
PKLLIYAASSLQSGVPSRFSGSGSGTDFTLTISSLQPEDFATY
YCQQTYSTPLTFGGGTKVEIKR
(SEQ ID NO: 8; CDRs underlined).

### mAb FIB-918-2

mAb FIB-918-2 includes a CDR-H1 including the amino acid sequence of GFTFSSYA (SEQ ID NO: 1); a CDR-H2 including the amino acid sequence of ISGSGGST (SEQ ID NO: 2); and a CDR-H3 including the amino acid sequence of AKDPRGSGRDDAFDI (SEQ ID NO: 20).

mAb FIB-918-2 includes a heavy chain variable domain having an amino acid sequence of

EVQLLESGGGLVQPGGSLRLSCAASGFTFSSYAMSWVRQAPGKGL
EWVSAISGSGGSTYYADSVKGRFTISRDNSKNTLYLQMNSLRAED
TAVYYCAKDPRGSGRDDAFDIWGQGTMVTVSS
(SEQ ID NO: 23; CDRs underlined).

mAb FIB-918-2 includes a CDR-L1 including the amino acid sequence of QTIGSY (SEQ ID NO: 21); a CDR-L2 including the amino acid sequence of GAS (SEQ ID NO: 22); and a CDR-L3 including the amino acid sequence of QQSYSTPFT (SEQ ID NO: 11).

mAb FIB-918-2 includes a light chain variable domain having an amino acid sequence of

NIQMTQSPSSLSASVGDRVTITCRASQTIGSYLNWYQQKPGTAPKL
LIYGASTSHTWVPSRFTGGGSGTEFTLTISSLQSEDFATYYCQQSY
STPFTFGGGTKLEIKR
(SEQ ID NO: 24; CDRs underlined).

### mAb FIB-918-3

mAb FIB-918-3 includes a CDR-H1 including the amino acid sequence of GFTFSSYS (SEQ ID NO: 14); a CDR-H2 including the amino acid sequence of ISSSSSTI (SEQ ID NO: 15); and a CDR-H3 including the amino acid sequence of ARGPDLSDYFDY (SEQ ID NO: 16).

mAb FIB-918-3 includes a heavy chain variable domain having an amino acid sequence of

EVQLVESGGGLVQPGGSLRLSCAASGFTFSSYSMNWVRQAPGKGLE
WVSYISSSSSTIYYADSVKGRFTISRDNAKNSLYLQMNSLRAEDTA
VYYCARGPDLSDYFDYWGRGTLVTVSS
(SEQ ID NO: 18; CDRsunderlined).

mAb FIB-918-3 includes a CDR-L1 including the amino acid sequence of QSISSY (SEQ ID NO: 4); a CDR-L2 including the amino acid sequence of AAS (SEQ ID NO: 5); and a CDR-L3 including the amino acid sequence of QQSYSTPLT (SEQ ID NO: 17).

mAb FIB-918-3 includes a light chain variable domain having an amino acid sequence of

DIQLTQSPSSLSASVGDRVTITCRASQSISSYLNWYQQKPGKAPK
LLIYAASSLQSGVPSRFSGSGSGTDFTLTISSLQPEDIATYYCQQ
SYSTPLTFGGGTKLEIKR
(SEQ ID NO: 19; CDRs underlined).

### mAb FIB-918-4

mAb FIB-918-4 includes a CDR-H1 including the amino acid sequence of GYTFTDYY (SEQ ID NO: 28); a CDR-H2 including the amino acid sequence of FDPEDGET (SEQ ID NO: 29); and a CDR-H3 including the amino acid sequence of ATLDYRGVVYFDY (SEQ ID NO: 30).

mAb FIB-918-4 includes a heavy chain variable domain having an amino acid sequence of

QVQLVQSGAEVKKPGATVKISCKVSGYTFTDYYMHWVRQAPGKGL
EWMGGFDPEDGETIYAQKFQGRVTMTEDTSTDTAYMELSSLRSED
TAVYYCATLDYRGVVYFDYWGQGTLVTVSS
(SEQ ID NO: 34; CDRs underlined).

mAb FIB-918-4 includes a CDR-L1 including the amino acid sequence of SGSIASNY (SEQ ID NO: 31); a CDR-L2 including the amino acid sequence of EDK (SEQ ID NO: 32); and a CDR-L3 including the amino acid sequence of QSYDSSNHWV (SEQ ID NO: 33).

mAb FIB-918-4 includes a light chain variable domain having an amino acid sequence of

NFMLTQPHSVSDSPGKTVTISCTGSSGSIASNYVQWYQQRPGSAPTTVI
YEDKRRPSGVPDRFIGSIDSSSNSASLTISGLRTEDEADYYCQSYDSSN
HWVFGGGTQLTVLG (SEQ ID NO: 35; CDRs underlined).

### mAb FIB-918-5

mAb FIB-918-5 includes a CDR-H1 including the amino acid sequence of GFTFSSYA (SEQ ID NO: 1); a CDR-H2 including the amino acid sequence of ISGSGGST (SEQ ID NO: 2); and a CDR-H3 including the amino acid sequence of AKDPTTMTTDAFDI (SEQ ID NO: 25).

mAb FIB-918-5 includes a heavy chain variable domain having an amino acid sequence of

EVQLVESGGGLVQPGGSLRLSCAASGFTFSSYAMSWVRQAPGKGLEWVS
AISGSGGSTYYADSVKGRFTISRDNSKNTLYLQMNSLRAEDTAVYYCAK
DPTTMTTDAFDIWGQGTMVTVSS (SEQ ID NO: 26; CDRs
underlined).

mAb FIB-918-5 includes a CDR-L1 including the amino acid sequence of QSISSY (SEQ ID NO: 4); a CDR-L2 including the amino acid sequence of AAS (SEQ ID NO: 5); and a CDR-L3 including the amino acid sequence of QQSYSTPFT (SEQ ID NO: 11).

mAb FIB-918-5 includes a light chain variable domain having an amino acid sequence of

DIQMTQSPSSLSASVGDRVTITCRASQSISSYLNWYQQKPGKAPKLLIY
AASSLQSGVPSRFSGSGSGTDFTLTISSLQPEDFATYYCQQSYSTPFTF
GPGTKLEIKR (SEQ ID NO: 27; CDRs underlined).

### mAb FIB-918-6

mAb FIB-918-6 includes a CDR-H1 including the amino acid sequence of GFTFSNAW (SEQ ID NO: 9); a CDR-H2 including the amino acid sequence of ISGSGGST (SEQ ID NO: 2); and a CDR-H3 including the amino acid sequence of ARDRGYSYSETSNDAFDI (SEQ ID NO: 10).

mAb FIB-918-6 includes a heavy chain variable domain having an amino acid sequence of

EVQLLESGGDLVKAGGSLRLSCAASGFTFSNAWMSWVRQAPGKGLEWVS
GISGSGGSTYYADSVKGRFTISRDNSKNTLYLQMNSLRAEDTAVYYCAR
DRGYSYSETSNDAFDIWGRGTLVTVSS (SEQ ID NO: 12; CDRs
underlined).

mAb FIB-918-6 includes a CDR-L1 including the amino acid sequence of QSISSY (SEQ ID NO: 4); a CDR-L2 including the amino acid sequence of AAS (SEQ ID NO: 5); and a CDR-L3 including the amino acid sequence of QQSYSTPFT (SEQ ID NO: 11).

mAb FIB-918-6 includes a light chain variable domain having an amino acid sequence of

DIQMTQSPSSLSASVGDRVTITCRASQSISSYLNWYQQKPGKAPKLLIY
AASSLQSGVPSRFSGSGSGTDFTLTISSLOPEDFATYYCQQSYSTPFTF
GPGTKLEIKR (SEQ ID NO: 13; CDRs underlined).

### mAb FIB-918-7

mAb FIB-918-7 includes a CDR-H1 including the amino acid sequence of GFMFDTHA (SEQ ID NO: 46); a CDR-H2 including the amino acid sequence of ISGSGGSI (SEQ ID NO: 47); and a CDR-H3 including the amino acid sequence of ARSGETAGTDYFDY (SEQ ID NO: 48).

mAb FIB-918-7 includes a heavy chain variable domain having an amino acid sequence of

EVQLVESGGGLVQPGGSLRLSCAASGFMFDTHAMSWVRQAPGKGLEWVS
SISGSGGSIYYADSVKGRFTISRDNSKNTLYLQMNSLRAEDTAVYYCAR
SGETAGTDYFDYWGQGTLVTVSS (SEQ ID NO: 51; CDRs.
underlined)

mAb FIB-918-7 includes a CDR-L1 including the amino acid sequence of QSVSSSY (SEQ ID NO: 49); a CDR-L2 including the amino acid sequence of GAS (SEQ ID NO: 22); and a CDR-L3 including the amino acid sequence of QQDYNSPYT (SEQ ID NO: 50).

mAb FIB-918-7 includes a light chain variable domain having an amino acid sequence of

EIVMTQSPATLSLSPGERATLSCRASQSVSSSYLSWYQQKPGQAPRLLI
YGASTRATGIPARFSGSGSGTDFTLTISSLQPEDFAVYYCQQDYNSPYT
FGQGTKVDIKR (SEQ ID NO: 52; CDRs underlined).

### mAb FIB-918-8

mAb FIB-918-8 includes a CDR-H1 including the amino acid sequence of GFTFSNAW (SEQ ID NO: 9); a CDR-H2 including the amino acid sequence of ISGSGGST (SEQ ID NO: 2); and a CDR-H3 including the amino acid sequence of AKQTVTSADDYFDY (SEQ ID NO: 43).

mAb FIB-918-8 includes a heavy chain variable domain having an amino acid sequence of

EVQLVESGGGVVRPGGPLRLSCAASGFTFSNAWMSWVRQAPGKGLEWVS
AISGSGGSTYYADSVKGRFTISRDNSKNTLYLQMNSLRAEDTAIYYCAK
QTVTSADDYFDYWGQGTLVTVSS (SEQ ID NO: 44; CDRs
underlined).

mAb FIB-918-8 includes a CDR-L1 including the amino acid sequence of QSISSY (SEQ ID NO: 4); a CDR-L2 including the amino acid sequence of AAS (SEQ ID NO: 5); and a CDR-L3 including the amino acid sequence of QQSYSTPFT (SEQ ID NO: 11).

mAb FIB-918-8 includes a light chain variable domain having an amino acid sequence of

DIQMTQSPSSLSASVGDRVTITCRASQSISSYLNWYQQKPGKAPKLLIY
AASSLQSGVPSRFSGSGSGTDFTLTISSLQPEDFATYYCQQSYSTPFTF
GPGTKLEIKR (SEQ ID NO: 45; CDRs underlined).

### mAb FIB-918-9

mAb FIB-918-9 includes a CDR-H1 including the amino acid sequence of GFTFSDYW (SEQ ID NO: 36); a CDR-H2 including the amino acid sequence of ISGSGGST (SEQ ID NO: 2); and a CDR-H3 including the amino acid sequence of AKDLLWAARDAFDI (SEQ ID NO: 37).

mAb FIB-918-9 includes a heavy chain variable domain having an amino acid sequence of

EVQLVESGGGLVQPGGSLRLSCLASGFTFSDYWMAWVRQAPGKGLEWVS
AISGSGGSTYYADSVKGRFTISRDNSKNTLYLQMNSLRAEDTAVYYCAK
DLLWAARDAFDIWGQGTLVTVSS (SEQ ID NO: 38; CDRs
underlined).

mAb FIB-918-9 includes a CDR-L1 including the amino acid sequence of QSISSY (SEQ ID NO: 4); a CDR-L2 including the amino acid sequence of AAS (SEQ ID NO: 5); and a CDR-L3 including the amino acid sequence of QQSYSTPFT (SEQ ID NO: 11).

mAb FIB-918-9 includes a light chain variable domain having an amino acid sequence of

DIQMTQSPSSLSASVGDRVTITCRASQSISSYLNWYQQKPGKAPKLLIY
AASSLQSGVPSRFSGSGSGTDFTLTISSLQPEDFATYYCQQSYSTPFTF
GPGTKLEIKR (SEQ ID NO: 39; CDRs underlined).

### mAb FIB-918-10

mAb FIB-918-10 includes a CDR-H1 including the amino acid sequence of GFTFSSYA (SEQ ID NO: 1); a CDR-H2 including the amino acid sequence of ISGSGGST (SEQ ID NO: 2); and a CDR-H3 including the amino acid sequence of AKDLLWAARDAFDI (SEQ ID NO: 37).

mAb FIB-918-10 includes a heavy chain variable domain having an amino acid sequence of

QVQLVESGGGLVQPGGSLRLSCAASGFTFSSYAMSWVRQAPGKGLEWVS
AISGSGGSTYYADSVKGRFTISRDNSKNTLYLQMNSLRAEDTAVYYCAK
DLLWAARDAFDIWGQGTLVTVSS (SEQ ID NO: 41; CDRs
underlined).

mAb FIB-918-10 includes a CDR-L1 including the amino acid sequence of QGINDF (SEQ ID NO: 40); a CDR-L2 including the amino acid sequence of AAS (SEQ ID NO: 5); and a CDR-L3 including the amino acid sequence of QQSYSTPLT (SEQ ID NO: 17).

mAb FIB-918-10 includes a light chain variable domain having an amino acid sequence of

AIQLTQSPSTLSASVGDRVTITCRASQGINDFLAWYQQKPGKAPKLLIY
AASSLQSGVPSRFSGSGSGTDFTLTISSLQPEDFATYYCQQSYSTPLTF
GGGTKVEIKR (SEQ ID NO: 42; CDRs underlined).

### Light Chain Variable Domain Sequences

This disclosure provides an antibody or antigen-binding fragment thereof that includes a light chain variable domain including an amino acid sequence with at least 70%, 75%, 80%, 85%, 86%, 87%, 88%, 89%, 90%, 91%, 92%, 93%, 94%, 95%, 96%, 97%, 98%, 99% or 100% sequence identity to the amino acid sequence of any one of SEQ ID NOs: 8, 13, 19, 24, 35, 39, 42, 45, and 52.

SEQ ID NO: 8
DIQMTQSPSSLSASVGDRVTITCRASQSISSYLNWYQQKPGKAPKLLIY
AASSLQSGVPSRFSGSGSGTDFTLTISSLQPEDFATYYCQQTYSTPLTF
GGGTKVEIKR
SEQ ID NO: 13
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPFTF
GPGTKLEIKR
SEQ ID NO: 19
DIQLTQSPSSLSASVGDRVTITCRASQSISSYLNWYQQKPGKAPKLLIY
AASSLQSGVPSRFSGSGSGTDFTLTISSLQPEDIATYYCQQSYSTPLTF
GGGTKLEIKR
SEQ ID NO: 24
NIQMTQSPSSLSASVGDRVTITCRASQTIGSYLNWYQQKPGTAPKLLIY
GASTSHTWVPSRFTGGGSGTEFTLTISSLQSEDFATYYCQQSYSTPFTF
GGGTKLEIKR
SEQ ID NO: 27
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPFTF
GPGTKLEIKR
SEQ ID NO: 35
NFMLTQPHSVSDSPGKTVTISCTGSSGSIASNYVQWYQQRPGSAPTTVI
YEDKRRPSGVPDRFIGSIDSSSNSASLTISGLRTEDEADYYCQSYDSSN
HWVFGGGTQLTVLG
SEQ ID NO: 39
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPFTF
GPGTKLEIKR
SEQ ID NO: 42
AIQLTQSPSTLSASVGDRVTITCRASQGINDFLAWYQQKPGKAPKLLIY
AASSLQSGVPSRFSGSGSGTDFTLTISSLQPEDFATYYCQQSYSTPLTF
GGGTKVEIKR
SEQ ID NO: 45
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPFTF
GPGTKLEIKR
SEQ ID NO: 52
EIVMTQSPATLSLSPGERATLSCRASQSVSSSYLSWYQQKPGQAPRLLI
YGASTRATGIPARFSGSGSGTDFTLTISSLQPEDFAVYYCQQDYNSPYT
FGQGTKVDIKR

### Heavy Chain Variable Domain Sequences

This disclosure provides an antibody or antigen-binding fragment thereof that includes a heavy chain variable domain including an amino acid sequence with at least 70%, 75%, 80%, 85%, 86%, 87%, 88%, 89%, 90%, 91%, 92%, 93%, 94%, 95%, 96%, 97%, 98%, 99% or 100% sequence identity to the amino acid sequence of any one of SEQ ID NOs: 7, 12, 18, 23, 26, 34, 38, 41, 44, and 51.

SEQ ID NO: 7
EVQLVESGGGLVQPGGSLRLSCAASGFTFSSYAMSWVRQAPGKGLEWVS
AISGSGGSTYYADSVKGRFTISRDNSKNTLYLQMNSLRAEDTAVYYCAK
DLDWSGHDAFDIWGQGTTVTVSS
SEQ ID NO: 12
EVQLLESGGDLVKAGGSLRLSCAASGFTFSNAWMSWVRQAPGKGLEWVS
GISGSGGSTYYADSVKGRFTISRDNSKNTLYLQMNSLRAEDTAVYYCAR
DRGYSYSETSNDAFDIWGRGTLVTVSS
SEQ ID NO: 18
EVQLVESGGGLVQPGGSLRLSCAASGFTFSSYSMNWVRQAPGKGLEWVS
YISSSSSTIYYADSVKGRFTISRDNAKNSLYLQMNSLRAEDTAVYYCAR
GPDLSDYFDYWGRGTLVTVSS
SEQ ID NO: 23
EVQLLESGGGLVQPGGSLRLSCAASGFTFSSYAMSWVRQAPGKGLEWVS
AISGSGGSTYYADSVKGRFTISRDNSKNTLYLQMNSLRAEDTAVYYCAK
DPRGSGRDDAFDIWGQGTMVTVSS
SEQ ID NO: 26
EVQLVESGGGLVQPGGSLRLSCAASGFTFSSYAMSWVRQAPGKGLEWVS
AISGSGGSTYYADSVKGRFTISRDNSKNTLYLQMNSLRAEDTAVYYCAK
DPTTMTTDAFDIWGQGTMVTVSS
SEQ ID NO: 34
QVQLVQSGAEVKKPGATVKISCKVSGYTFTDYYMHWVRQAPGKGLEWMG
GFDPEDGETIYAQKFQGRVTMTEDTSTDTAYMELSSLRSEDTAVYYCAT
LDYRGVVYFDYWGQGTLVTVSS
SEQ ID NO: 38
EVQLVESGGGLVQPGGSLRLSCLASGFTFSDYWMAWVRQAPGKGLEWVS
AISGSGGSTYYADSVKGRFTISRDNSKNTLYLQMNSLRAEDTAVYYCAK
DLLWAARDAFDIWGQGTLVTVSS
SEQ ID NO: 41
QVQLVESGGGLVQPGGSLRLSCAASGFTFSSYAMSWVRQAPGKGLEWVS
AISGSGGSTYYADSVKGRFTISRDNSKNTLYLQMNSLRAEDTAVYYCAK
DLLWAARDAFDIWGQGTLVTVSS
SEQ ID NO: 44
EVQLVESGGGVVRPGGPLRLSCAASGFTFSNAWMSWVRQAPGKGLEWVS
AISGSGGSTYYADSVKGRFTISRDNSKNTLYLQMNSLRAEDTAIYYCAK
QTVTSADDYFDYWGQGTLVTVSS
SEQ ID NO: 51
EVQLVESGGGLVQPGGSLRLSCAASGFMFDTHAMSWVRQAPGKGLEWVS
SISGSGGSIYYADSVKGRFTISRDNSKNTLYLQMNSLRAEDTAVYYCAR
SGETAGTDYFDYWGQGTLVTVSS

Antibodies described herein include fully human, humanized, primatized, and chimeric antibodies. Additionally, antibodies described herein include fully human, humanized, primatized, and chimeric antibodies that contain one or more, or all, of the CDR-H1, CDR-H2, CDR-H3, CDR-L1, CDR-L2, and CDR-L3 sequences described herein in which one or more, or all, of the CDR sequences exhibits at least 70% sequence identity (e.g., 75%, 80%, 85%, 90%, 95%, 96%, 97%, 98%, 99%, or 100% sequence identity) to the corresponding CDR sequence of an antibody described herein (e.g., any one of mAbs FIB-918-1, FIB-918-2, FIB-918-3, FIB-918-4, FIB-918-5, FIB-918-6, FIB-918-7, FIB-918-8, FIB-918-9, or FIB-918-10).

In some embodiments, the antibody or antigen binding fragment is a humanized antibody or antigen-binding fragment that contains one or more, or all, of the CDR-H1, CDR-H2, CDR-H3, CDR-L1, CDR-L2, and CDR-L3 sequences described herein in which one or more, or all, of the CDR sequences have at least 70% sequence identity (e.g., 75%, 80%, 85%, 90%, 95%, 96%, 97%, 98%, 99%, or 100% sequence identity) to the corresponding CDR sequence of a humanized antibody described herein (e.g., any one of mAbs FIB-918-1, FIB-918-2, FIB-918-3, FIB-918-4, FIB-918-5, FIB-918-6, FIB-918-7, FIB-918-8, FIB-918-9, or FIB-918-10).

Antibodies and antibody fragments described herein further include fully human, humanized, primatized, and chimeric antibodies that contain one or more, or all, of the CDR-H1, CDR-H2, CDR-H3, CDR-L1, CDR-L2, and CDR-L3 sequences in which one or more, or all, of the CDR sequences contains one or more (for instance, up to 3) amino acid substitutions (e.g., one or more conservative amino acid substitutions) relative to the corresponding CDR sequence of an antibody described herein. For example, antibodies described herein can be generated by incorporating any one or more of the CDR sequences of an antibody described herein into the framework regions (e.g., FW1, FW2, FW3, and FW4) of a human antibody.

As an example, one strategy that can be used to design humanized antibodies described herein is to align the sequences of the heavy chain variable region and light chain variable region of an antibody described herein with the heavy chain variable region and light chain variable region of a consensus human antibody. Consensus human antibody heavy chain and light chain sequences are known in the art (see e.g., the “VBASE” human germline sequence database; see also Kabat, et al., Sequences of Proteins of Immunological Interest, Fifth Edition, U.S. Department of Health and Human Services, NIH Publication No. 91-3242, 1991; Tomlinson et al., J. Mol. Biol. 227:776-98, 1992; and Cox et al, Eur. J. Immunol. 24:827-836, 1994; the disclosure of which is incorporated herein by reference). In this way, the variable domain framework residues and CDRs can be identified by sequence alignment (see Kabat, supra). One can substitute, for example, one or more of the CDRs of the consensus human antibody with the corresponding CDR(s) of an antibody described herein, in order to produce a humanized antibody.

Exemplary variable domains of a consensus human antibody include the heavy chain variable domain are identified in U.S. Pat. No. 6,054,297; the disclosure of which is incorporated herein by reference. These amino acid substitutions can be made, for example, by recombinant expression of polynucleotides encoding the heavy and light chains of a humanized antibody in a host cell using methods known in the art or described herein.

Similarly, this strategy can also be used to produce primatized antibodies, as one can substitute, for example, one or more, or all, of the CDRs of a primate antibody consensus sequence with, for example, one or more, or all, of the CDRs of an antibody described herein. Consensus primate antibody sequences known in the art (see e.g., U.S. Pat. Nos. 5,658,570; 5,681,722; and 5,693,780; the disclosures of each of which are incorporated herein by reference).

In some embodiments, it may be desirable to import particular framework residues in addition to CDR sequences from an antibody, such as an antibody described herein, into the heavy and/or light chain variable domains of a human antibody. For instance, U.S. Pat. No. 6,054,297 identifies several instances when it may be advantageous to retain certain framework residues from a particular antibody heavy chain or light chain variable region in the resulting humanized antibody. In some embodiments, framework residues may engage in non-covalent interactions with the antigen and thus contribute to the affinity of the antibody for the target antigen. In some embodiments, individual framework residues may modulate the conformation of a CDR, and thus indirectly influence the interaction of the antibody with the antigen. Certain framework residues may form the interface between VH and VL domains and may therefore contribute to the global antibody structure. In some cases, framework residues may constitute functional glycosylation sites (e.g., Asn-X-Ser/Thr) which may dictate antibody structure and antigen affinity upon attachment to carbohydrate moieties. In cases such as those described above, it may be beneficial to retain certain framework residues of an antibody described herein in, e.g., a humanized or primatized antagonistic antibody or antigen-binding fragment thereof, as various framework residues may promote high epitope affinity and improved biochemical activity of the antibody or antigen-binding fragment thereof.

Antibodies described herein also include antibody fragments, Fab domains, F(ab′) molecules, F(ab′)₂ molecules, single-chain variable fragments (scFvs), tandem scFv fragments, diabodies, triabodies, dual variable domain immunoglobulins, multi-specific antibodies, bispecific antibodies, and heterospecific antibodies that contain one or more, or all, of the CDRs of an antibody described herein, or one or more, or all, of the CDR-H1, CDR-H2, CDR-H3, CDR-L1, CDR-L2, and CDR-L3 sequences in which one or more, or all, of the CDR sequences exhibits at least 70% sequence identity (e.g., 75%, 80%, 85%, 90%, 95%, 96%, 97%, 98%, 99%, or 100% sequence identity) to the corresponding CDR sequence of an antibody described herein. Antibodies described herein further include fully human, humanized, primatized, and chimeric antibodies that contain one or more, or all, of the CDR-H1, CDR-H2, CDR-H3, CDR-L1, CDR-L2, and CDR-L3 sequences in which one or more, or all, of the CDR sequences contains one or more (for instance, up to 3) amino acid substitutions (e.g., one or more conservative amino acid substitutions) relative to the corresponding CDR sequence of an antibody described herein. These molecules can be expressed recombinantly, e.g., by incorporating polynucleotides encoding these proteins into expression vectors for transfection in a eukaryotic or prokaryotic cell using techniques described herein or known in the art, or synthesized chemically, e.g., by solid phase peptide synthesis methods described herein or known in the art.

Polypeptides described herein additionally include antibody-like scaffolds that contain, for example, one or more, or all, of the CDRs of an antibody described herein, or one or more, or all, of the CDR-H1, CDR-H2, CDR-H3, CDR-L1, CDR-L2, and CDR-L3 sequences in which one or more, or all, of the CDR sequences exhibits at least 70% sequence identity (e.g., 75%, 80%, 85%, 90%, 95%, 96%, 97%, 98%, 99%, or 100% sequence identity) to the corresponding CDR sequence of an antibody described herein or contains one or more (for instance, up to 3) amino acid substitutions (e.g., one or more conservative amino acid substitutions) relative to the corresponding CDR sequence of an antibody described herein. Examples of antibody-like scaffolds include proteins that contain a tenth fibronectin type III domain (¹⁰Fn3), which contains BC, DE, and FG structural loops analogous to canonical antibodies. The tertiary structure of the ¹⁰Fn3 domain resembles that of the variable region of the IgG heavy chain, and one of skill in the art can graft, e.g., one or more, or all, of the CDR sequences of an antibody or antigen-binding fragment thereof described herein or sequences having at least 70% sequence identity (e.g., 75%, 80%, 85%, 90%, 95%, 97%, 99%, or 100% sequence identity) to any one or more of these CDR sequences or sequences containing amino acid substitutions, such as conservative or nonconservative amino acid substitutions (e.g., up to 3 amino acid substitutions) relative to one or more of these CDR sequences onto the fibronectin scaffold by replacing residues of the BC, DE, and FG loops of ¹⁰Fn3 with residues of the corresponding CDR sequence of an antibody or antigen-binding fragment thereof described herein. This can be achieved by recombinant expression of a modified ¹⁰Fn3 domain in a prokaryotic or eukaryotic cell (e.g., using the vectors and techniques described herein). Examples of using the ¹⁰Fn3 domain as an antibody-like scaffold for the grafting of CDRs from antibodies onto the BC, DE, and FG structural loops are reported in WO 2000/034784, WO 2009/142773, WO 2012/088006, and U.S. Pat. No. 8,278,419; the disclosures of each of which are incorporated herein by reference.

### II. Nucleic Acids and Expression Systems

Antibodies or antigen-binding fragments thereof described herein can be prepared by any of a variety of established techniques. For instance, an anti-ITGA11 antibody or antigen-binding fragment thereof described herein can be prepared by recombinant expression of immunoglobulin light and heavy chain genes in a host cell. To express an antibody recombinantly, a host cell can be transfected with one or more recombinant expression vectors carrying DNA fragments encoding the immunoglobulin light and heavy chains of the antibody such that the light and heavy chains are expressed in the host cell and, optionally, secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Molecular Cloning; A Laboratory Manual, Second Edition (Sambrook, Fritsch and Maniatis (eds), Cold Spring Harbor, N. Y., 1989), Current Protocols in Molecular Biology (Ausubel et al., eds., Greene Publishing Associates, 1989), and in U.S. Pat. No. 4,816,397; the disclosures of each of which are incorporated herein by reference.

### Vectors for Expression of Anti-ITGA11 Antibodies

Viral genomes provide a rich source of vectors that can be used for the efficient delivery of exogenous genes into the genome of a cell (e.g., a eukaryotic or prokaryotic cell). Viral genomes are particularly useful vectors for gene delivery because the polynucleotides contained within such genomes are typically incorporated into the genome of a target cell by generalized or specialized transduction. These processes occur as part of the natural viral replication cycle, and do not require added proteins or reagents in order to induce gene integration. Examples of viral vectors include a retrovirus, adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses useful for delivering polynucleotides encoding antibody light and heavy chains or antibody fragments described herein include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030); the disclosures of each of which are incorporated herein by reference.

### Genome Editing Techniques

In addition to viral vectors, a variety of additional methods have been developed for the incorporation of genes, e.g., those encoding antibody light and heavy chains, single-chain polypeptides, single-chain variable fragments (scFvs), tandem scFvs, Fab domains, F(ab′)₂ domains, diabodies, and triabodies, among others, such as those described herein, into the genomes of target cells for polypeptide expression. One such method that can be used for incorporating polynucleotides encoding antibodies or fragments thereof, such as those described herein, into prokaryotic or eukaryotic cells includes transposons. Transposons are polynucleotides that encode transposase enzymes and contain a polynucleotide sequence or gene of interest flanked by excision sites at the 5′ and 3′ positions. Once a transposon has been delivered into a cell, expression of the transposase gene commences and results in active enzymes that cleave the gene of interest from the transposon. This activity is mediated by the site-specific recognition of transposon excision sites by the transposase. In some embodiments, these excision sites may be terminal repeats or inverted terminal repeats. Once excised from the transposon, the gene of interest can be integrated into the genome of a prokaryotic or eukaryotic cell by transposase-catalyzed cleavage of similar excision sites that exist within nuclear genome of the cell. This allows the gene encoding an antibody or fragment or domain thereof to be inserted into the cleaved nuclear DNA at the excision sites, and subsequent ligation of the phosphodiester bonds that join the gene of interest to the DNA of the prokaryotic or eukaryotic cell genome completes the incorporation process. In some embodiments, the transposon may be a retrotransposon, such that the gene encoding the antibody is first transcribed to an RNA product and then reverse-transcribed to DNA before incorporation in the prokaryotic or eukaryotic cell genome. Exemplary transposon systems include the piggybac transposon (described in detail in WO 2010/085699) and the sleeping beauty transposon (described in detail in US20050112764); the disclosures of each of which are incorporated herein by reference.

Another useful method for the integration of nucleic acid molecules encoding antibodies or fragments thereof, such as those described herein, into the genome of a prokaryotic or eukaryotic cell is the clustered regularly interspaced short palindromic repeats (CRISPR)/Cas system, which is a system that originally evolved as an adaptive defense mechanism in bacteria and archaea against infection by viruses. The CRISPR/Cas system consists of palindromic repeat sequences within plasmid DNA and an associated Cas9 nuclease. This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas9 nuclease to this site. In this manner, highly site-specific cas9-mediated DNA cleavage can be engendered in a foreign polynucleotide because the interaction that brings cas9 within close proximity of the target DNA molecule is governed by RNA: DNA hybridization. As a result, one can theoretically design a CRISPR/Cas system to cleave any target DNA molecule of interest. This technique has been exploited in order to edit eukaryotic genomes (Hwang et al., Nat. Biotech., 31:227-229, 2013) and can be used as an efficient means of site-specifically editing eukaryotic or prokaryotic genomes in order to cleave DNA prior to the incorporation of a polynucleotide encoding an antibody or fragment thereof described herein. The use of CRISPR/Cas to modulate gene expression has been described in U.S. Pat. No. 8,697,359, the disclosure of which is incorporated herein by reference.

Alternative methods for site-specifically cleaving genomic DNA prior to the incorporation of a polynucleotide encoding an antibody or fragment thereof, such as those described herein, include the use of zinc finger nucleases and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. Zinc finger nucleases and TALENs for use in genome editing applications are described in Urnov et al. (Nat. Rev. Genet., 11:636-646, 2010); and in Joung et al., (Nat. Rev. Mol. Cell. Bio. 14:49-55, 2013); incorporated herein by reference. Additional genome editing techniques that can be used to incorporate polynucleotides encoding antibodies described herein into the genome of a prokaryotic or eukaryotic cell include the use of ARCUS™ meganucleases that can be rationally designed so as to site-specifically cleave genomic DNA. The use of these enzymes for the incorporation of polynucleotides encoding antibodies or fragments thereof described herein into the genome of a prokaryotic or eukaryotic cell is particularly advantageous in view of the structure-activity relationships that have been established for such enzymes. Single-chain meganucleases can thus be modified at certain amino acid positions in order to create nucleases that selectively cleave DNA at desired locations. These single-chain nucleases have been described extensively, e.g., in U.S. Pat. Nos. 8,021,867 and 8,445,251; the disclosures of each of which are incorporated herein by reference.

### Polynucleotide Sequence Elements

To express antibodies or fragments thereof, such as those described herein, polynucleotides encoding partial or full-length light and heavy chains, e.g., polynucleotides that encode a one or more, or all, of the CDR sequences of an antibody or antigen-binding fragment thereof described herein, can be inserted into expression vectors such that the genes are operatively linked to transcriptional and translational control sequences. The expression vector and expression control sequences are chosen to be compatible with the expression host cell used. Polynucleotides encoding the light chain gene and the heavy chain of an antibody or fragment thereof can be inserted into separate vectors, or, optionally, both polynucleotides can be incorporated into the same expression vector using established techniques described herein or known in the art.

In addition to polynucleotides encoding the heavy and light chains of an antibody (or a polynucleotide encoding a single-chain polypeptide, an antibody fragment, such as a scFv molecule, or a construct described herein), the recombinant expression vectors described herein may carry regulatory sequences that control the expression of the antibody chain genes in a host cell. The design of the expression vector, including the selection of regulatory sequences, may depend on such factors as the choice of the host cell to be transformed or the level of expression of protein desired. For instance, suitable regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. Viral regulatory elements, and sequences thereof, are described in detail, for instance, in U.S. Pat. Nos. 5,168,062, 4,510,245, and 4,968,615, the disclosures of each of which are incorporated herein by reference.

In addition to the antibody chain genes and regulatory sequences, the recombinant expression vectors described herein can carry additional sequences, such as sequences that regulate replication of the vector in host cells (e.g., origins of replication) and selectable marker genes. A selectable marker gene facilitates selection of host cells into which the vector has been introduced (see e.g., U.S. Pat. Nos. 4,399,216, 4,634,665 and 5,179,017). For example, typically the selectable marker gene confers resistance to cytotoxic drugs, such as G418, puromycin, blasticidin, hygromycin or methotrexate, to a host cell into which the vector has been introduced. Suitable selectable marker genes include the dihydrofolate reductase (DHFR) gene (for use in DHFR″ host cells with methotrexate selection/amplification) and the neo gene (for G418 selection). In order to express the light and heavy chains of an antibody or fragment thereof, the expression vector(s) containing polynucleotides encoding the heavy and light chains can be transfected into a host cell by standard techniques.

### Host Cells for Expression of an Anti-ITGA11 Antibody or a Fragment Thereof

It is possible to express the antibodies of fragments thereof described herein in either prokaryotic or eukaryotic host cells. In certain embodiments, expression of antibodies or fragments thereof is performed in eukaryotic cells, e.g., mammalian host cells, for optimal secretion of a properly folded and immunologically active antibody. Exemplary mammalian host cells for expressing the recombinant antibodies or antigen-binding fragments thereof described herein include Chinese Hamster Ovary (CHO cells) (including DHFR CHO cells, described in Urlaub and Chasin (1980, Proc. Natl. Acad. Sci. USA 77:4216-4220), used with a DHFR selectable marker, e.g., as described in Kaufman and Sharp (1982, Mol. Biol. 159:601-621), NSO myeloma cells, COS cells, 293 cells, and SP2/0 cells. Additional cell types that may be useful for the expression of antibodies and fragments thereof include bacterial cells, such as BL-21 (DE3) E. coli cells, which can be transformed with vectors containing foreign DNA according to established protocols. Additional eukaryotic cells that may be useful for expression of antibodies include yeast cells, such as auxotrophic strains of S. cerevisiae, which can be transformed and selectively grown in incomplete media according to established procedures known in the art. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown.

Antibodies or antigen-binding fragments thereof can be recovered from the culture medium using standard protein purification methods. Host cells can also be used to produce portions of intact antibodies, such as Fab fragments or scFv molecules. Also included herein are methods in which the above procedure is varied according to established protocols known in the art. For example, it can be desirable to transfect a host cell with DNA encoding either the light chain or the heavy chain (but not both) of an antibody or fragment thereof described herein in order to produce an antigen-binding fragment of the antibody.

Once an antibody or fragment thereof described herein has been produced by recombinant expression, it can be purified by any method known in the art, such as a method useful for purification of an immunoglobulin molecule, for example, by chromatography (e.g., ion exchange, affinity, and sizing column chromatography), centrifugation, differential solubility, or by any other standard technique for the purification of proteins. Further, the antibody or fragment thereof described herein or fragments thereof can be fused to heterologous polypeptide sequences described herein or otherwise known in the art to facilitate purification or to produce therapeutic conjugates.

Once isolated, an antibody or fragment thereof can, if desired, be further purified, e.g., by high performance liquid chromatography (see, e.g., Fisher, Laboratory Techniques in Biochemistry and Molecular Biology (Work and Burdon, eds., Elsevier, 1980); incorporated herein by reference), or by gel filtration chromatography, such as on a SUPERDEX™ 75 column (Pharmacia Biotech AB, Uppsala, Sweden).

### III. Generation and Purification of Anti-ITGA11 Antibodies or Fragments Thereof

Antigens including an epitope that corresponds to a portion of ITGA11 may be used to generate, for example, monoclonal, polyclonal, chimeric, humanized, or recombinant ITGA11-specific antibodies. Methods include the immunological methods described by Kohler and Milstein (Nature 256:495-497, 1975 and Eur. J. Immunol. 6:511-519, 1976) and Campbell (“Monoclonal Antibody Technology, The Production and Characterization of Rodent and Human Hybridomas,” in Burdon et al., Eds., Laboratory Techniques in Biochemistry and Molecular Biology, Volume 13, Elsevier Science Publishers, Amsterdam, 1985), as well as by the recombinant DNA method described by Huse et al. (Science 246:1275-1281, 1989).

Briefly, an antigen may, in combination with an adjuvant, be administered to a host animal (e.g., rabbits, mice, rats, goats, guinea pigs, hamsters, horses, and sheep, as well as non-human primates). The administration of such antigens may be accomplished by any of a variety of methods, including, but not limited to, subcutaneous or intramuscular injection. Once administered, the results of antibody titers produced in the host animal are monitored, which may be conducted by any of a variety of techniques well-known in the art (e.g., routine bleeds), with the antisera being isolated (e.g., via centrifugation) and thereafter screened for the presence of antibodies having a binding affinity for, e.g., ITGA11. Screening for the desired antibody may be accomplished by techniques including, e.g., radioimmunoassays, ELISA, sandwich immunoassays, immunoradiometric assays, gel diffusion precipitation reactions, in situ immunoassays (e.g., using colloidal gold, enzymatic, or radioisotope labels), Western blots, precipitation reactions, agglutination assays (e.g., gel agglutination assays or hemagglutination assays), complement fixation assays, immunofluorescence assays, protein A assays, and immunoelectrophoresis assays.

The resultant antisera derived from the host animal may be affinity purified to derive the antibodies for the present disclosure. The antisera may be purified via conventional techniques, such as the introduction of the antisera onto a separation column. The antigens of the present disclosure may be immobilized on the column to isolate and purify antibodies. The column may then be washed to remove antibodies not having specificity for the antigen immobilized on the column, with the remaining antibody ultimately being eluted from the column. The isolated antibody may then be stored per conventional practices known to those skilled in the art.

Established procedures for immunizing primates are known in the art (see, e.g., WO 1986/6004782; incorporated herein by reference). Immunization represents a robust method of producing monoclonal antibodies by exploiting the antigen specificity of B lymphocytes. For example, monoclonal antibodies can be prepared by the Kohler-Millstein procedure (described, e.g., in EP 0110716; incorporated herein by reference), wherein spleen cells from a non-human animal (e.g., a primate) administer peptide with an antigenic peptide. A clonally-expanded B lymphocyte produced by immunization can be isolated from the serum of the animal and subsequently fused with a myeloma cell in order to form a hybridoma. Hybridomas are particularly useful agents for antibody production, as these immortalized cells can provide a lasting supply of an antigen-specific antibody. Antibodies from such hybridomas can subsequently be isolated using techniques known in the art, e.g., by purifying the antibodies from the cell culture medium by affinity chromatography.

Alternatively, antibody libraries (e.g., naive antibody libraries, synthetic antibody libraries, semi-synthetic antibody libraries, or combinatorial libraries) may be screened for the identification of antibodies. Such libraries are commercially available from a number of sources (e.g., Cambridge Antibody, Cambridge, United Kingdom, Genetastix Corporation, Pacific Northwest Laboratory, Richland,

Washington, and MorphoSys AG, Munich, Germany (e.g., HuCal GOLD)). See, e.g., U.S. Pat. Nos. 6,696,248; 6,706,484; 6,828,422; and 7,264,963, hereby incorporated by reference.

Screening of an antibody library may be performed by using one of the methods known to one of skill in the art including, e.g., phage-display, selectively infective phage, polysome technology, and assay systems for enzymatic activity or protein stability. Antibodies having the desired property can be identified, for example, by sequencing of the corresponding nucleic acid sequence, by amino acid sequencing, or by mass spectrometry. Optimization is performed by replacing sub-sequences with different sequences (e.g., random sequences) and then repeating the screening step one or more times. The antibodies may be screened for, e.g., optimized affinity or specificity for a target molecule, optimized expression yields, optimized stability, or optimized solubility.

Antibodies of the present disclosure recognize and specifically bind to ITGA11. In some embodiments, the Kd between the antibody and ITGA11 is, for example, at least about 10⁻⁴ M, 10⁻⁵ M, 10 6 M, 10⁻⁷ M, 10⁻⁸ M, 10⁻⁹ M, 10⁻¹⁰ M, 10⁻¹¹ M, or 10⁻¹² M or greater.

### IV. Anti-ITGA11 Antibody Conjugates

It may be desirable to conjugate the antibody or fragment thereof to a second molecule, e g., to modulate the activity of the antibody in vivo or for diagnostic purposes. Antibodies or antigen-binding fragments thereof can be conjugated to other molecules at either the N-terminus or C-terminus of a light or heavy chain of the antibody using any one of a variety of established conjugation strategies that are well-known in the art. Examples of pairs of reactive functional groups that can be used to covalently tether an antibody or antigen-binding fragment thereof to another molecule include, without limitation, thiol pairs, carboxylic acids and amino groups, ketones and amino groups, aldehydes and amino groups, thiols and alpha, beta-unsaturated moieties (such as maleimides or dehydroalanine), thiols and alpha-halo amides, carboxylic acids and hydrazides, aldehydes and hydrazides, and ketones and hydrazides.

Antibodies or antigen-binding fragments thereof can be covalently appended directly to another molecule by chemical conjugation as described. Alternatively, fusion proteins containing an antibody or antigen-binding fragment thereof can be expressed recombinantly from a cell (e.g., a eukaryotic cell or prokaryotic cell). This can be accomplished, for example, by incorporating a polynucleotide encoding the fusion protein into the nuclear genome of a cell (e.g., using techniques described herein or known in the art). Optionally, antibodies and fragments thereof described herein can be joined to a second molecule by forming a covalent bond between the antibody and a linker. This linker can then be subsequently conjugated to another molecule, or the linker can be conjugated to another molecule prior to ligation to the antibody or antigen-binding fragment thereof. Examples of linkers that can be used for the formation of a conjugate include polypeptide linkers, such as those that contain naturally occurring or non-naturally occurring amino acids. In some embodiments, it may be desirable to include D-amino acids in the linker, as these residues are not present in naturally-occurring proteins and are thus more resistant to degradation by endogenous proteases. Fusion proteins containing polypeptide linkers can be made using chemical synthesis techniques, such as those described herein, or through recombinant expression of a polynucleotide encoding the fusion protein in a cell (e.g., a prokaryotic or eukaryotic cell). Linkers can be prepared using a variety of strategies that are well known in the art, and depending on the reactive components of the linker, can be cleaved by enzymatic hydrolysis, photolysis, hydrolysis under acidic conditions, hydrolysis under basic conditions, oxidation, disulfide reduction, nucleophilic cleavage, or organometallic cleavage (Leriche et al., Bioorg. Med. Chem., 20:571-582, 2012).

An antibody or antigen-binding fragment thereof described herein can be conjugated to, admixed with, or administered separately from a therapeutic agent.

### Labeled Anti-ITGA11 Antibodies or Antigen-Binding Fragments Thereof

In some embodiments, antibodies or antigen-binding fragments thereof described herein are conjugated to another molecule (e.g., an epitope tag) for the purpose of purification or detection. Examples of such molecules that are useful in protein purification include those that present structural epitopes capable of being recognized by a second molecule. This is a common strategy that is employed in protein purification by affinity chromatography, in which a molecule is immobilized on a solid support and exposed to a heterogeneous mixture containing a target protein conjugated to a molecule capable of binding the immobilized compound. Examples of epitope tag molecules that can be conjugated to an antibodies or antigen-binding fragments thereof described herein for the purposes of molecular recognition include, without limitation, maltose-binding protein, glutathione-S-transferase, a poly-histidine tag, a FLAG-tag, a myc-tag, human influenza hemagglutinin (HA) tag, biotin, streptavidin. Conjugates containing the epitopes presented by these molecules are capable of being recognized by such complementary molecules as maltose, glutathione, a nickel-containing complex, an anti-FLAG antibody, an anti-myc antibody, an anti-HA antibody, streptavidin, or biotin, respectively. For example, one can purify an antibody or fragment thereof described herein that has been conjugated to an epitope tag from a complex mixture of other proteins and biomolecules (e.g., DNA, RNA, carbohydrates, phospholipids, etc.) by treating the mixture with a solid phase resin containing an complementary molecule that can selectively recognize and bind the epitope tag of the antibody or fragment thereof. Examples of solid phase resins include agarose beads, which are compatible with purifications in aqueous solution.

An antibody or antigen-binding fragment thereof described herein can also be covalently appended to a fluorescent molecule, e.g., to detect the antibody or antigen-binding fragment thereof by fluorimetry and/or by direct visualization using fluorescence microscopy. Exemplary fluorescent molecules that can be conjugated to antibodies described herein include green fluorescent protein, cyan fluorescent protein, yellow fluorescent protein, red fluorescent protein, phycoerythrin, allophycocyanin, hoescht, 4′,6-diamidino-2-phenylindole (DAPI), propidium iodide, fluorescein, coumarin, rhodamine, tetramethylrhodamine, and cyanine. Additional examples of fluorescent molecules suitable for conjugation to antibodies described herein are well-known in the art and have been described in detail in, e.g., U.S. Pat. Nos. 7,417,131 and 7,413,874, each of which is incorporated by reference herein.

Antibodies or antigen-binding fragments thereof containing a fluorescent molecule are particularly useful for monitoring the cell-surface localization properties of antibodies and fragments thereof described herein. For instance, one can expose cultured mammalian cells to antibodies or antigen-binding fragments thereof described herein that have been covalently conjugated to a fluorescent molecule and subsequently analyze these cells using conventional fluorescent microscopy techniques known in the art. Confocal fluorescent microscopy is a particularly powerful method for determining cell-surface localization of tagged antibodies, as individual planes of a cell can be analyzed in order to distinguish antibodies or fragments thereof that have been internalized into a cell's interior, e.g., by receptor-mediated endocytosis, from those that are bound to the external face of the cell membrane. Additionally, cells can be treated with an antibody conjugated to a fluorescent molecule that emits visible light of a particular wavelength (e.g., fluorescein, which fluoresces at about 535 nm) and an additional fluorescent molecule that is known to localize to a particular site on the cell surface and that fluoresces at a different wavelength (e.g., a molecule that localizes to CD25 and that fluoresces at about 599 nm). The resulting emission patterns can be visualized by confocal fluorescence microscopy and the images from these two wavelengths can be merged in order to reveal information regarding the location of the antibody or antigen-binding fragment thereof on the cell surface with respect to other receptors.

Bioluminescent proteins can also be incorporated into a fusion protein for the purposes of detection and visualization of antibodies or fragments thereof. Bioluminescent proteins, such as Luciferase and aequorin, emit light as part of a chemical reaction with a substrate (e.g., luciferin and coelenterazine). Exemplary bioluminescent proteins suitable for use as a diagnostic sequence and methods for their use are described in, e.g., U.S. Pat. Nos. 5,292,658, 5,670,356, 6,171,809, and 7,183,092, each of which is herein incorporated by reference. Antibodies or antigen-binding fragments thereof labeled with bioluminescent proteins are a useful tool for the detection of antibodies described herein following an in vitro assay. For instance, the presence of an antibody that has been conjugated to a bioluminescent protein can be detected among a complex mixture of additional proteins by separating the components of the mixture using gel electrophoresis methods known in the art (e.g., native gel analysis) and subsequently transferring the separated proteins to a membrane in order to perform a Western blot. Detection of the antibody among the mixture of other proteins can be achieved by treating the membrane with an appropriate Luciferase substrate and subsequently visualizing the mixture of proteins on film using established protocols.

An antibody or antigen-binding fragment thereof described herein can also be conjugated to a molecule including a radioactive nucleus, such that an antibody or fragment thereof described herein can be detected by analyzing the radioactive emission pattern of the nucleus. Alternatively, an antibody or fragment thereof can be modified directly by incorporating a radioactive nucleus within the antibody during the preparation of the protein. Radioactive isotopes of methionine (³⁵S), nitrogen (¹⁵N), or carbon (¹³C) can be incorporated into antibodies or fragments thereof described herein by, e.g., culturing bacteria in media that has been supplemented with nutrients containing these isotopes. Optionally, tyrosine derivatives containing a radioactive halogen can be incorporated into an antibody by, e.g., culturing bacterial cells in media supplemented with radiolabeled tyrosine. It has been shown that tyrosine functionalized with a radioactive halogen at the C2 position of the phenol system are rapidly incorporated into elongating polypeptide chains using the endogenous translation enzymes in vivo (U.S. Pat. No. 4,925,651; incorporated herein by reference). The halogens include fluorine, chlorine, bromine, iodine, and astatine. Additionally, an antibody can be modified following isolation and purification from cell culture by functionalizing polypeptides described herein with a radioactive isotope. The halogens represent a class of isotopes that can be readily incorporated into a purified protein by aromatic substitution at tyrosine or tryptophan, e.g., via reaction of one or more of these residues with an electrophilic halogen species. Examples of radioactive halogen isotopes include ¹⁸F, ⁷⁵Br, ⁷⁷Br, ¹²²I, ^{123I}, ^{124I}, ¹²⁵I, ¹²⁹I, ¹³¹I, or ²¹¹At.

Another alternative strategy for the incorporation of a radioactive isotope is the covalent attachment of a chelating group to the antibody or fragment thereof, or construct. Chelating groups can be covalently appended to an antibody or fragment thereof by attachment to a reactive functional group, such as a thiol, amino group, alcohol, or carboxylic acid. The chelating groups can then be modified to contain any of a variety of metallic radioisotopes, including, without limitation, such radioactive nuclides as ¹²⁵I, ⁶⁷Ga, ¹¹¹ In, ⁹⁹Tc, ¹⁶⁹Yb, ¹⁸⁶Re, ¹²³I, ¹²⁴I, ¹²⁵I, ¹³¹I, ⁹⁹mTc, ¹¹¹ In, ⁶⁴Cu, ⁶⁷Cu, ¹⁸⁶Re, ¹⁸⁸Re, ¹⁷⁷Lu, ⁹⁰Y, ⁷⁷As, ⁷²As, ⁸⁶Y, ⁸⁹Zr, ²¹¹At, ²¹²Bi, ²¹³Bi, or ²²⁵Ac.

In some embodiments, it may be desirable to covalently conjugate the antibodies or fragments thereof described herein with a chelating group capable of binding a metal ion from heavy elements or rare earth ions, such as Gd³⁺, Fe³⁺, Mn³⁺, or Cr²⁺. Conjugates containing chelating groups that are coordinated to such paramagnetic metals are useful as in MRI imaging applications. Paramagnetic metals include, but are not limited to, chromium (III), manganese (II), iron (II), iron (III), cobalt (II), nickel (II), copper (II), praseodymium (III), neodymium (III), samarium (III), gadolinium (III), terbium (III), dysprosium (III), holmium (III), erbium (III), and ytterbium (III). In this way, antibodies can be detected by MRI spectroscopy. For instance, one can administer antibodies or fragments thereof conjugated to chelating groups bound to paramagnetic ions to a mammalian subject (e.g., a human patient) in order to monitor the distribution of the antibody following administration. This can be achieved by administration of the antibody to a patient by any of the administration routes described herein, such as intravenously, and subsequently analyzing the location of the administered antibody by recording an MRI of the patient according to established protocols. An antibody or antigen-binding fragment thereof can additionally be conjugated to other molecules for the purpose of improving the solubility and stability of the protein in aqueous solution. Examples of such molecules include PEG, PSA, bovine serum albumin (BSA), and human serum albumin (HSA), among others. For instance, one can conjugate an antibody to carbohydrate moieties in order to evade detection of the antibody or fragment thereof by the immune system of the patient receiving treatment. This process of hyperglycosylation reduces the immunogenicity of therapeutic proteins by sterically inhibiting the interaction of the protein with B cell receptors in circulation. Alternatively, antibodies or fragments thereof can be conjugated to molecules that prevent clearance from human serum and improve the pharmacokinetic profile of antibodies described herein. Exemplary molecules that can be conjugated to or inserted within an antibody or antigen-binding fragment thereof described herein so as to attenuate clearance and improve the pharmacokinetic profile of these antibodies and fragments include salvage receptor binding epitopes. These epitopes are found within the Fc region of an IgG immunoglobulin and have been shown to bind Fc receptors and prolong antibody half-life in human serum. The insertion of salvage receptor binding epitopes into antibodies or fragments thereof can be achieved, e.g., as described in U.S. Pat. No. 5,739,277; incorporated herein by reference.

### V. Methods of Treatment

Anti-ITGA11 antibodies or antigen-binding fragments thereof described herein can be used to treat a patient suffering from a disorder associated with ITGA11 (e.g., a disorder that is effected by the inhibition of ITGA11), for example a fibrotic disorder, an inflammatory disorder, or a cancer. Anti-ITGA11 antibodies or antigen-binding fragments thereof can be administered to a mammalian subject, such as a human, suffering from a fibrotic disorder, an inflammatory disorder, or a cancer.

### Fibrotic Disorders

Fibrosis is a common response to a range of tissue insults that may lead to organ dysfunction. Diseases that are characterized by pathological fibrosis, and which can be treated using the methods and compositions of the invention, include, without limitation, hepatic fibrosis (e.g., fibrosis associated with cirrhosis (e.g., alcohol-induced cirrhosis, viral-induced cirrhosis, post-hepatitis C cirrhosis, and primary biliary cirrhosis), schistosomiasis, cholangitis (e.g., sclerosing cholangitis), and autoimmune-induced hepatitis), kidney fibrosis (e.g., tubulointerstitial fibrosis, scleroderma, diabetic nephritis, and glomerular nephritis), dermal fibrosis (e.g., scleroderma, hypertrophic and keloid scarring, nephrogenic fibrosing dermatopathy, and burns), myelofibrosis, neurofibromatosis, fibroma, intestinal fibrosis, and fibrotic adhesions resulting from surgical procedures), heart fibrosis (e.g., fibrosis associated with myocardial infarction), vascular fibrosis (e.g., fibrosis associated with postangioplasty arterial restenosis and atherosclerosis), ocular fibrosis (e.g., fibrosis associated with post-cataract surgery, proliferative vitreoretinopathy, and retro-orbital fibrosis), bone marrow fibrosis (e.g., idiopathic myelofibrosis and drug-induced myelofibrosis), pulmonary fibrosis (e.g., pulmonary interstitial fibrosis), glomerulonephritis, heart failure (ischemic and non-ischemic), scleroderma, excessive scar tissue post-surgery or device insertion, trauma or burns, progressive kidney disease, valvular heart disease, hypertensive heart disease, articular and periarticular fibrosis, myelofibrosis, ocular/vitreous fibrosis, intestinal fibrosis and stricture, peritoneal and retro-peritoneal fibrosis, pancreatic fibrosis, nephrogenic systemic fibrosis, primary sclerosing cholangitis, and the elaboration of pathological matrix also has a role in fibroproliferative tumor progression and metastasis.

### Inflammatory Disorders

Inflammation can be classified as either acute or chronic. Acute inflammation is the initial response of the body to harmful stimuli and is achieved by the increased movement of plasma and leukocytes (especially granulocytes) from the blood into the injured tissues. A series of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation and is characterized by simultaneous destruction and healing of the tissue from the inflammatory process. Diseases that are characterized by pathological inflammation, and which can be treated using the methods and compositions of the invention, include, without limitation, asthma (e.g., allergic asthma, exercise-induced asthma, aspirin sensitive/exacerbated asthma, atopic asthma, severe asthma, mild asthma, moderate to severe asthma, corticosteroid naïve asthma, chronic asthma, corticosteroid resistant asthma, corticosteroid refractory asthma, newly diagnosed and untreated asthma, asthma due to smoking, asthma uncontrolled on corticosteroids, and the like), airway inflammation, airway hyperreactivity, airway hyperresponsiveness, rhinosinusitis, rhinosinusitis with polyps, nasal polyposis, arthritis (e.g., osteoarthritis, rheumatoid arthritis, collagen-induced arthritis, arthritic joints as a result of injury, etc.), eosinophilic inflammation, mast cell-mediated inflammatory diseases, sepsis, septic shock, seronegative enthesopathy and arthropathy (SEA) syndrome, osteoporosis, eosinophilic esophagitis, scleroderma, dermatitis, atopic dermatitis, allergic rhinitis, bullous pemphigoid, urticaria (e.g., chronic urticaria), cartilage inflammation, polymyalgia rheumatic, polyarteritis nodossa, Wegener's granulomatosis, Behcet's disease, myolitis, polymyolitis, dermatomyolitis, dermatomyositis, vasculitis, arteritis, diabetic nephropathy, interstitial cystitis, graft versus host disease (GVHD), gastrointestinal inflammatory conditions (e.g., inflammatory bowel disease (IBD), ulcerative colitis (UC), Crohn's disease (CD), colitis (e.g., colitis caused by environmental insults (e.g., caused by or associated with a therapeutic regimen, such as chemotherapy, radiation therapy, and the like), infectious colitis, ischemic colitis, collagenous or lymphocytic colitis, necrotizing enterocolitis, colitis in conditions such as chronic granulomatous disease or celiac disease, food allergies, gastritis, infectious gastritis or enterocolitis (e.g., Helicobacter pylori-infected chronic active gastritis), and other forms of gastrointestinal inflammation caused by an infectious agent), and inflammatory pulmonary conditions (e.g., chronic obstructive pulmonary disease (COPD), eosinophilic pulmonary inflammation, infection-induced pulmonary conditions (including those associated with viral (e.g., influenza, parainfluenza, rotavirus, human metapneumovirus, and respiratory syncytial virus), bacterial, fungal (e.g., Aspergillus), parasitic, or prion infection, allergen-induced pulmonary conditions, pollutant-induced pulmonary conditions (e.g., asbestosis, silicosis, or berylliosis), gastric aspiration-induced pulmonary conditions, immune dysregulation, inflammatory conditions with genetic predisposition such as cystic fibrosis, physical trauma-induced pulmonary conditions (e.g., ventilator injury), emphysema, bronchitis, sarcoidosis, histiocytosis, lymphangiomyomatosis, acute lung injury, acute respiratory distress syndrome, chronic lung disease, bronchopulmonary dysplasia, pneumonia (e.g., community-acquired pneumonia, nosocomial pneumonia, ventilator-associated pneumonia, viral pneumonia, bacterial pneumonia, and severe pneumonia), airway exacerbations, and acute respiratory distress syndrome (ARDS)).

### Cancer

Cancer is a condition characterized by a population of cells exhibiting unregulated cell growth. ITGA11 signaling may contribute to human carcinogenesis, including invasion and metastasis. Cancers which can be treated using the methods and compositions of the invention, include, without limitation, breast cancer, colorectal cancer, hepatic cancer, kidney cancer, liver cancer, lung cancer, pancreatic cancer, gastrointestinal cancer, melanoma, ovarian cancer, prostate cancer, cervical cancer, bladder cancer, glioblastoma, head and neck cancer, and cholangiocarcinoma. In some embodiments, the methods further include administering to the subject at least one additional anti-cancer agent.

### VI. Pharmaceutical Compositions

Pharmaceutical compositions containing an anti-ITGA11 antibody or antigen-binding fragment thereof described herein can be prepared using methods known in the art. Particularly, anti-ITGA11 antibodies or antigen-binding fragments thereof that can be incorporated into pharmaceutical compositions of the disclosure include an anti-ITGA11 antibody or antigen-binding fragment thereof that contains one or more, or all, of the CDR sequences of an antibody or antigen-binding fragment thereof described herein, such as a human, humanized, or chimeric variant of an anti-ITGA11 described herein.

Pharmaceutical compositions described herein may contain an antibody or antigen-binding fragment thereof described herein in combination with one or more pharmaceutically acceptable excipients. For instance, pharmaceutical compositions described herein can be prepared using, e.g., physiologically acceptable carriers, excipients or stabilizers (Remington's Pharmaceutical Sciences 16th edition, Osol, A. Ed. (1980); incorporated herein by reference), and in a desired form, e.g., in the form of lyophilized formulations or aqueous solutions. The compositions can also be prepared so as to contain the active agent at a desired concentration. For example, a pharmaceutical composition described herein may contain at least 10% (e.g., 10%, 20%, 30%, 40%, 50%, 60%, 70%, 80%, 90%, 95%, 97%, 98%, 99%, 99.5%, 99.9%, or 100%) active agent by weight (w/w).

Additionally, an active agent that can be incorporated into a pharmaceutical formulation can itself have a desired level of purity. For example, an antibody or antigen-binding fragment thereof described herein may be characterized by a certain degree of purity after isolating the antibody from cell culture media or after chemical synthesis, e.g., of a single-chain antibody fragment (e.g., scFv) by established solid phase peptide synthesis methods or native chemical ligation as described herein. An antibody or antigen-binding fragment thereof described herein may be at least 10% pure prior to incorporating the antibody into a pharmaceutical composition (e.g., 10%, 20%, 30%, 40%, 50%, 60%, 70%, 80%, 90%, 95%, 97%, 98%, 99%, 99.5%, 99.9%, 99.99%, or 100% pure).

Pharmaceutical compositions of antibodies or antigen-binding fragments thereof described herein can be prepared for storage as lyophilized formulations or aqueous solutions by mixing the antibody having the desired degree of purity with optional pharmaceutically acceptable carriers, excipients or stabilizers typically employed in the art, e.g., buffering agents, stabilizing agents, preservatives, isotonifiers, non-ionic detergents, antioxidants, and other miscellaneous additives. See, e.g., Remington's Pharmaceutical Sciences, 16th edition (Osol, ed. 1980; incorporated herein by reference). Such additives must be nontoxic to the recipients at the dosages and concentrations employed.

### Buffering Agents

Buffering agents help to maintain the pH in the range which approximates physiological conditions. They can be present at concentration ranging from about 2 mM to about 50 mM. Suitable buffering agents for use with antibodies or antigen-binding fragments thereof described herein include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers {e.g., fumaric acid-monosodium fumarate mixture, fumaric acid-disodium fumarate mixture, monosodium fumarate-disodium fumarate mixture, etc.), gluconate buffers (e.g., gluconic acid-sodium gluconate mixture, gluconic acid-sodium hydroxide mixture, gluconic acid-potassium gluconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers {e.g., acetic acid-sodium acetate mixture, acetic acid-sodium hydroxide mixture, etc.). Additionally, phosphate buffers, histidine buffers and trimethylamine salts such as Tris can be used.

### Preservatives

Preservatives can be added to a composition described herein to retard microbial growth and can be added in amounts ranging from 0.2%-1% (w/v). Suitable preservatives for use with antibodies or antigen-binding fragments thereof described herein include phenol, benzyl alcohol, meta-cresol, methyl paraben, propyl paraben, octadecyldimethylbenzyl ammonium chloride, benzalconium halides {e.g., chloride, bromide, and iodide), hexamethonium chloride, and alkyl parabens such as methyl or propyl paraben, catechol, resorcinol, cyclohexanol, and 3-pentanol. Isotonifiers sometimes known as “stabilizers” can be added to ensure isotonicity of liquid compositions described herein and include polhydric sugar alcohols, for example trihydric or higher sugar alcohols, such as glycerin, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins; hydrophilic polymers, such as polyvinylpyrrolidone monosaccharides, such as xylose, mannose, fructose, glucose; disaccharides such as lactose, maltose, sucrose and trisaccharides such as raffinose; and polysaccharides such as dextran. Stabilizers can be present in the range from 0.1 to 10,000 weights per part of weight active protein.

### Detergents

Non-ionic surfactants or detergents (also known as “wetting agents”) can be added to help solubilize the therapeutic agent as well as to protect the therapeutic protein against agitation-induced aggregation, which also permits the formulation to be exposed to shear surface stressed without causing denaturation of the protein. Suitable non-ionic surfactants include polysorbates (20, 80, etc.), polyoxamers (184, 188 etc.), Pluronic polyols, polyoxyethylene sorbitan monoethers (TWEEN®-20, TWEEN®-80, etc.). Non-ionic surfactants can be present in a range of about 0.05 mg/ml to about 1.0 mg/mL, for example about 0.07 mg/ml to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

### Other Pharmaceutical Carriers

Alternative pharmaceutically acceptable carriers that can be incorporated into a pharmaceutical composition described herein may include dextrose, sucrose, sorbitol, mannitol, starch, rubber arable, potassium phosphate, arginate, gelatin, potassium silicate, microcrystalline cellulose, polyvinylpyrrolidone, cellulose, water, syrups, methyl cellulose, methylhydroxy benzoate, propylhydroxy benzoate, talc, magnesium stearate, and mineral oils, but not limited to. A composition containing antibody described herein may further include a lubricant, a humectant, a sweetener, a flavoring agent, an emulsifier, a suspending agent, and a preservative. Details of suitable pharmaceutically acceptable carriers and formulations can be found in Remington's Pharmaceutical Sciences (19th ed., 1995), which is incorporated herein by reference.

### VII. Routes of Administration and Dosing

An anti-ITGA11 antibody or antigen-binding fragment thereof described herein can be administered to a mammalian subject (e.g., a human) by a variety of routes, such as orally, transdermally, subcutaneously, intranasally, intravenously, intramuscularly, intraocularly, intratumorally, parenterally, topically, intrathecally and intracerebroventricularly, for the treatment of, e.g., the diseases and conditions described herein (e.g., a fibrotic disorder, and inflammatory disorder, or a cancer). The most suitable route for administration in any given case will depend on the particular polypeptide administered, the patient, pharmaceutical formulation methods, administration methods (e.g., administration time and administration route), the patient's age, body weight, sex, severity of the diseases being treated, the patient's diet, and the patient's excretion rate.

A physician having ordinary skill in the art can readily determine an effective amount of an anti-ITGA11 antibody or antigen-binding fragment thereof for administration to a mammalian subject (e.g., a human) in need thereof. For example, a physician could start prescribing doses of an antibody described herein at levels lower than that required in order to achieve the desired therapeutic effect and gradually increase the dosage until the desired effect is achieved. Alternatively, a physician may begin a treatment regimen by administering an antibody or antigen-binding fragment thereof described herein at a high dose and subsequently administering progressively lower doses until a therapeutic effect is achieved. In general, a suitable daily dose of an antibody or antigen-binding fragment thereof will be an amount of the compound which is the lowest dose effective to produce a therapeutic effect. An antigen-binding fragment thereof described herein may be administered, e.g., by injection, such as by intravenous, intramuscular, intraperitoneal, or subcutaneous injection, optionally proximal to the site of the target tissue. A daily dose of a therapeutic composition of an antibody described herein may be administered as a single dose or as two, three, four, five, six or more doses administered separately at appropriate intervals throughout the day, week, month, or year, or as needed, optionally, in unit dosage forms. While it is possible for an antibody described herein to be administered alone, it may also be administered as a pharmaceutical formulation in combination with excipients, carriers, and optionally, additional therapeutic agents.

The effective dose of an anti-ITGA11 antibody or antigen-binding fragment thereof described herein can range, for instance, from about 0.0001 to about 100 mg/kg of body weight per single (e.g., bolus) administration, multiple administrations or continuous administration (e.g., a continuous infusion), or to achieve a serum concentration of 0.0001-5000 g/mL serum concentration per single (e.g., bolus) administration, multiple administrations or continuous administration (e.g., continuous infusion), or any effective range or value therein depending on the condition being treated, the route of administration and the age, weight, and condition of the subject. In certain embodiments, each dose can range from about 0.0001 mg to about 500 mg/kg of body weight. For instance, a pharmaceutical composition described herein may be administered in a daily dose in the range of 0.001-100 mg/kg (body weight). The dose may be administered one or more times (e.g., 2-10 times) per day, week, month, or year to a mammalian subject (e.g., a human) in need thereof.

Anti-ITGA11 antibodies or antigen-binding fragments thereof can be administered to a patient by way of a continuous intravenous infusion or as a single bolus administration. The antibodies or antigen-binding fragments thereof may be administered to a patient in an amount of, for example, from 0.01 ug to about 5 g in a volume of, for example, from 10 uL to 10 mL. The antibodies or antigen-binding fragments thereof may be administered to a patient over the course of several minutes to several hours. For example, the antibodies or antigen-binding fragments thereof described herein may be administered to a patient over the course of from 5 minutes to 5 hours, such as over the course of 5 minutes, 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 80 minutes, 90 minutes, 95 minutes, 100 minutes, 105 minutes, 110 minutes, 115 minutes, 120 minutes, 125 minutes, 130 minutes, 135 minutes, 140 minutes, 145 minutes, 150 minutes, 155 minutes, 160 minutes, 165 minutes, 170 minutes, 175 minutes, 180 minutes, 185 minutes, 190 minutes, 195 minutes, 200 minutes, 205 minutes, 210 minutes, 215 minutes, 220 minutes, 225 minutes, 230 minutes, 235 minutes, 240 minutes, 245 minutes, 250 minutes, 255 minutes, 260 minutes, 265 minutes, 270 minutes, 275 minutes, 280 minutes, 285 minutes, 290 minutes, 295 minutes, or 300 minutes, or more.

When anti-ITGA11 antibodies or antigen-binding fragments thereof are administered to a patient in combination with an additional therapeutic agent, the antibody or antigen-binding fragment thereof and the additional therapeutic agent may be co-administered to the patient, for example, by way of a continuous intravenous infusion or bolus administration of the first agent, followed by a continuous intravenous infusion or bolus administration of the second agent. The administration of the two agents may occur concurrently. Alternatively, the administration of the antibody or antigen-binding fragment thereof may precede or follow the administration of the additional therapeutic agent. In some embodiments, administration of the second agent (e.g., the antibody or antigen-binding fragment thereof) commences within from about 5 minutes to about 4 weeks, or more, of the end of the administration of the first agent (e.g., the additional therapeutic agent). For example, administration of the second agent may commence within about 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, 7 days, 2 weeks, 3 weeks, 4 weeks, or more, of the end of the administration of the first agent.

Therapeutic compositions can be administered with medical devices known in the art. For example, in an embodiment, a therapeutic composition described herein can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in conjunction with the compositions and methods described herein include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

### VIII. Kits Containing Anti-ITGA11 Antibodies or Antigen-Binding Fragments Thereof

Also included herein are kits that contain anti-ITGA11 antibodies or antigen-binding fragments thereof. The kits provided herein may contain any of the antibodies or antigen-binding fragments thereof described above, as well as any of the polynucleotides encoding these polypeptides, vectors containing these polynucleotides, or cells engineered to express and secrete antibodies described herein (e.g., prokaryotic or eukaryotic cells).

A kit described herein may include reagents that can be used to produce the compositions described herein (e.g., an anti-ITGA11 antibody or antigen-binding fragment thereof). Optionally, kits described herein may include reagents that can induce the expression of an antibody or antigen-binding fragment thereof within cells (e.g., mammalian cells), such as doxycycline or tetracycline. In other cases, a kit described herein may contain a compound capable of binding and detecting a fusion protein that contains an antibody or antigen-binding fragment thereof and an epitope tag. For instance, in such cases a kit described herein may contain maltose, glutathione, a nickel-containing complex, an anti-FLAG antibody, an anti-myc antibody, an anti-HA antibody, biotin, or streptavidin.

Kits described herein may also include reagents that are capable of detecting an anti-ITGA11 antibody or antigen-binding fragment thereof directly. Examples of such reagents include secondary antibodies that selectively recognize and bind particular structural features within the Fc region of an antibody or antigen-binding fragment thereof described herein. Kits described herein may contain secondary antibodies that recognize the Fc region of an antibody or antigen-binding fragment thereof and that are conjugated to a fluorescent molecule. These antibody-fluorophore conjugates provide a tool for analyzing the localization of antibodies or antigen-binding fragments thereof, e.g., in a particular tissue or cultured mammalian cell using established immunofluorescence techniques. In some embodiments, kits described herein may include additional fluorescent compounds that exhibit known sub-cellular localization patterns. These reagents can be used in combination with another antibody-fluorophore conjugate, e.g., one that specifically recognizes a different receptor on the cell surface in order to analyze the localization of an antibody or antigen-binding fragment thereof relative to other cell-surface proteins.

Kits described herein may also contain a reagent that can be used for the analysis of a patient's response to treatment by administration of antibodies or antigen-binding fragments thereof described herein. For instance, kits described herein may include an antibody or antigen-binding fragment thereof and one or more reagents that can be used to determine the quantity of T-reg cells in a blood sample withdrawn from a subject (e.g., a human) that is undergoing treatment with an antibody described herein. Kits may contain, e.g., antibodies that selectively bind cell-surface antigens presented by T-reg cells, such as CD4 and CD25. Optionally, these antibodies may be labeled with a fluorescent dye, such as fluorescein or tetramethylrhodamine, in order to facilitate analysis of T-reg cells by fluorescence-activated cell sorting (FACS) methods known in the art. Kits described herein may optionally contain one or more reagents that can be used to quantify tumor-reactive T lymphocytes in order to determine the effectiveness of an antibodies or antigen-binding fragments thereof in restoring tumor-infiltrating lymphocyte proliferation. For instance, kits described herein may contain an antibody that selectively binds cell-surface markers on the surface of a cytotoxic T cell, such as CD8 or CD3. Optionally, these antibodies may be labeled with fluorescent molecules so as to enable quantitation by FACS analysis.

A kit described herein may also contain one or more reagents useful for determining the affinity and selectivity of an antibody or antigen-binding fragment thereof described herein for one or more peptides derived from ITGA11. For instance, a kit may contain an anti-ITGA11 antibody or antigen-binding fragment thereof and one or more reagents that can be used in an ELISA assay to determine the K_{D} of an antibody described herein for one or more peptides that present a ITGA11 epitope in a conformation similar to that of the epitope in the native protein. A kit may contain, e.g., a microtiter plate containing wells that have been previously conjugated to avidin, and may contain a library of ITGA11-derived peptides, each of which conjugated to a biotin moiety. Such a kit may optionally contain a secondary antibody that specifically binds to the Fc region of an antibody or antigen-binding fragment thereof described herein, and the secondary antibody may be conjugated to an enzyme (e.g., horseradish peroxidase) that catalyzes a chemical reaction that results in the emission of luminescent light.

Kits described herein may also contain an antibody or antigen-binding fragment thereof described herein and a reagent that can be conjugated to such an antibody, including those previously described (e.g., a cytotoxic agent, a fluorescent molecule, a bioluminescent molecule, a molecule containing a radioactive isotope, a molecule containing a chelating group bound to a paramagnetic ion, etc). These kits may additionally contain instructions for how the conjugation of an antibody or antigen-binding fragment thereof described herein to a second molecule, such as those described above, can be achieved.

A kit described herein may also contain a vector containing a polynucleotide that encodes an antibody or antigen-binding fragment thereof, such as any of the vectors described herein. Alternatively, a kit may include mammalian cells (e.g., CHO cells) that have been genetically altered to express and secrete antibodies or antigen-binding fragments thereof or fragments thereof from the nuclear genome of the cell. Such a kit may also contain instructions describing how expression of the antibody or antigen-binding fragment thereof from a polynucleotide can be induced, and may additionally include reagents (such as, e.g., doxycycline or tetracycline) that can be used to promote the transcription of these polynucleotides. Such kits may be useful for the manufacture of antibodies or antigen-binding fragments thereof described herein.

Other kits described herein may include tools for engineering a prokaryotic or eukaryotic cell (e.g., a CHO cell or a BL21 (DE3) E. coli cell) so as to express and secrete an antibody or antigen-binding fragment thereof described herein from the nuclear genome of the cell. For example, a kit may contain CHO cells stored in an appropriate media and optionally frozen according to methods known in the art. The kit may also provide a vector containing a polynucleotide that encodes a nuclease (e.g., such as the CRISPER/Cas, zinc finger nuclease, TALEN, ARCUS™ nucleases described herein) as well as reagents for expressing the nuclease in the cell. The kit can additionally provide tools for modifying the polynucleotide that encodes the nuclease so as to enable one to alter the DNA sequence of the nuclease in order to direct the cleavage of a specific target DNA sequence of interest. Examples of such tools include primers for the amplification and site-directed mutagenesis of the polynucleotide encoding the nuclease of interest. The kit may also include restriction enzymes that can be used to selectively excise the nuclease-encoding polynucleotide from the vector and subsequently re-introduce the modified polynucleotide back into the vector once the user has modified the gene. Such a kit may also include a DNA ligase that can be used to catalyze the formation of covalent phosphodiester linkages between the modified nuclease-encoding polynucleotide and the target vector. A kit described herein may also provide a polynucleotide encoding an antibody or antigen-binding fragment thereof, as well as a package insert describing the methods one can use to selectively cleave a particular DNA sequence in the genome of the cell in order to incorporate the polynucleotide encoding an antibody or antigen-binding fragment thereof into the genome at this site. Optionally, the kit may provide a polynucleotide encoding a fusion protein that contains an antibody or antigen-binding fragment thereof or fragment thereof and an additional polypeptide, such as, e.g., those described herein.

### Examples

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1. Generation and Purification of Anti-ITGA11 Antibodies

Anti-ITGA11 antibodies were generated using a naïve human scFv library generated by IONTAS with a diversity of 10\hat{ }10 unique clones. Two rounds of phage display selection were performed on this library with a total of 8 selection strands that included selection on various combinations of recombinant soluble human ITGA11β1, recombinant murine ITGA11β1, cell expressed human ITGA11β1 and cell expressed murine ITGA11β1. Deselection was also included with cell expressed human ITGA2β1 and recombinant ITGA2β1. Each selection strand was performed in the presence or absence of 1 mM MnCl₂ for a total of 8 selection strands that included outputs with kappa light chains and 8 with lambda light chains. Following the selection and deselection with a final round on cell expressed ITGA11, all selection outputs showed enrichment following round 2 of selection.

Following the isolation of the scFv phage display selection outputs, there were converted to mammalian display human IgG1 format. These converted outputs were grouped into 4 mammalian display library and library size determined using methods known in the art. These libraries were generated for mammalian display transfections and selection in HEK293 cells. One hundred million HEK293 cells were transfected with 20 ug of the library DNA mixture and one day post transfection, cells from each transfection were incubated with anti-hu Fc-PE labeled antibody to assess the transient expression level cell-surface IgG1. In the first round of pre mammalian cell display (MCD), the transfected libraries were subject to MACS (Militenyi Midi MACS separator) sorting to enrich for cells expressing high levels of human IgG1 (this was detected by anti-PE magnetic beds bound to anti Fc-PE bound to cells expressing human IgG1. The results for each of the mammalian display libraries showed enrichment of binders for human ITGA11β1. In round 1 of MCD all MCD libraries were incubated with 0.1 nM biotinylated human ITGA11β1 and sorted using FACS. Unlabeled human ITGA10β1 was included to reduce the number of sorted clones that bound it. Thirty millions cells were sorted per library. From 1.5 to 4.9% of the top human ITGA11β1 binders at 0.1 nM were collected and placed into cell culture for a second round of sorting. This process was repeated in a round two sort increasing the stringency of human ITGA11 binding to enrich for higher affinity binders and including biotinylated ITGA10 as a specificity sort as well.

Mammalian display output cell populations from the round two sort were sub-clone into a soluble human IgG4 (S228P) expression vector. Plasmid DNA preparations for output population expression vectors were prepared and grown up in E. coli using methods known in the art. Colonies were picked from all outputs and distributed across 10×96 deep well plates in 1.2 ml LB media and kanamycin and incubated overnight at 37° C. with shaking. Each plate contained 90 antibody clones, a positive control and a negative control antibody and 2 blank wells. Plasmid DNA for each well of all 10 plates was extracted and purified using MagBind kit. Purified plasmid DNA was used to transfect ExpiHEK293 cells for expression of each soluble antibody clone. The conditioned media containing each antibody was used for primary screening experiments. All human IgG4 antibody clone containing supes from the 10 plates were assessed for binding to CHO expressing human ITGA11β1 and to recombinant human ITGA11β1 protein by Elisa. Of the 900 clones screened 61 unique clones bound to CHO human ITGA11β1 cells while showing minimal binding to CHO human ITGA10β1 or human ITGA2β1. These 61 antibody clones were transiently expressed in Expi293 cells in 24 deep well plates the antibodies purified using Protein A affinity chromatography following standard methods known by those in the art.

The 61 purified antibody hits were then assessed functionally using a tiered flow scheme where their ability to bind to cells expressing human ITGA11β1 at 34 nM and for their ability to block human ITGA11β1 binding to collagen at 68 nM was measured. Of the 61 hits, twenty antibodies that blocked the binding of collagen I to human ITGA11β1 by at least 50% at 68 nM were identified. All of these antibodies demonstrated measurable binding to human ITGA11B and to cynomolgus ITGA11β1, and 17 of 20 bound to murine ITGA11β1.

### Example 2. Characterization of Binding of Anti-ITGA11 Antibodies

Antibody binding to C2C12 cells expressing human ITGA11 or CHO cells expressing murine ITGA11 was performed by incubating the cells with the antibodies at a concentration of 34 nM for 1 hour at 4° C. in binding medium (PBS, 10% normal goat serum, 2% rabbit serum, and 1% BSA). Cells were washed in binding medium and binding was detected using goat-anti-human Fc BV421 (Jackson ImmunoLabs). After washing the cells were resuspended in binding medium and binding was assessed by flow cytometry. 226 antibodies were screened and the binding characteristics of 10 antibodies are presented below. The results are provided in Table 2 and are expressed as mean fluorescence intensity (MFI).

TABLE 2
Relative binding of human anti-ITGA11 antibodies
to human ITGA11 and mouse ITGA11

| | Binding Human ITGA11 | Binding Murine ITGA11 |
|---|---|---|
| Antibody | (MFI) | (MFI) |
| FIB-918-1 | 3506 | 2555 |
| FIB-918-6 | 7885 | 9407 |
| FIB-918-3 | 5225 | 1519 |
| FIB-918-2 | 30386 | 12584 |
| FIB-918-5 | 4271 | 2744 |
| FIB-918-4 | 15726 | 4566 |
| FIB-918-9 | 1452 | 2030 |
| FIB-918-10 | 10567 | 8572 |
| FIB-918-8 | 41466 | 32906 |
| FIB-918-7 | 7868 | 21272 |
| Control IgG4 | 557 | 513 |

### Example 3. Anti-ITGA11 Antibodies Inhibit Binding of Collagen to Cell-Expressed ITGA11

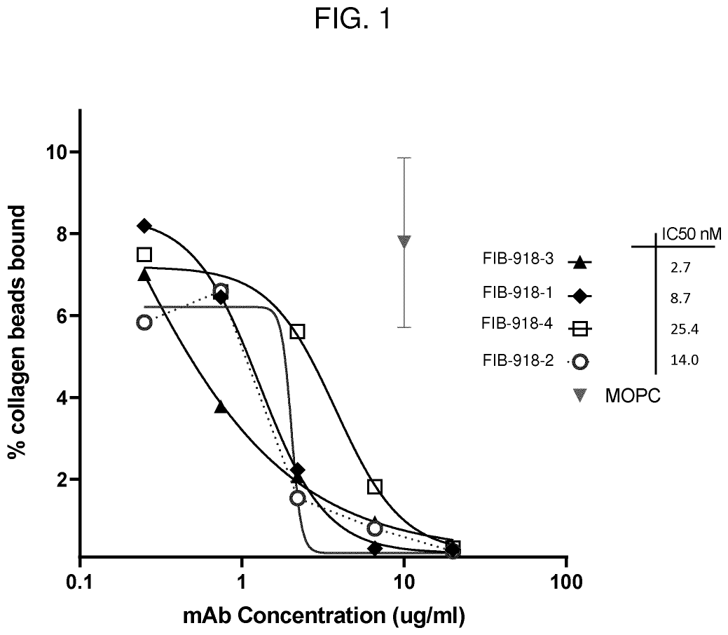

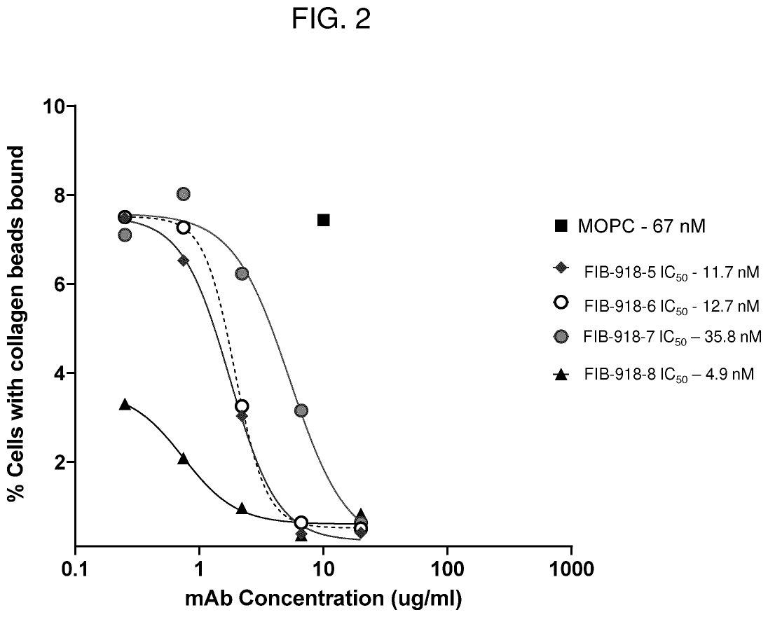

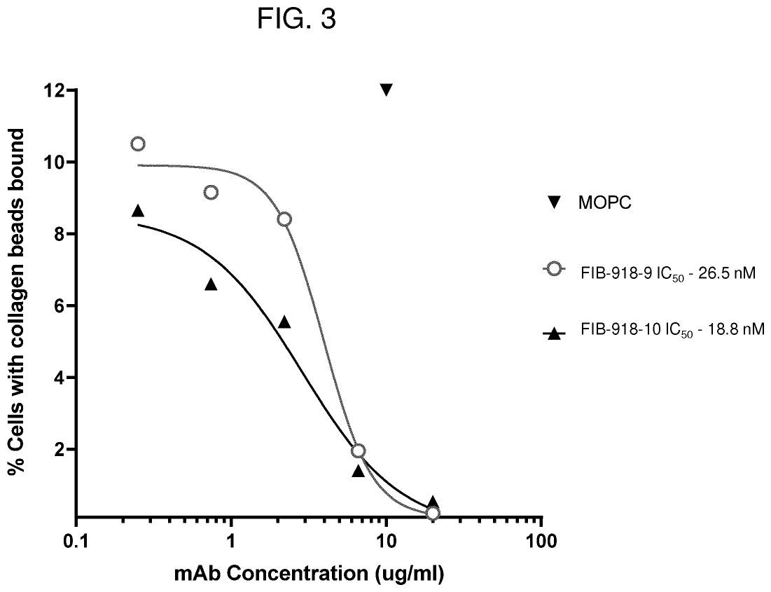

Collagen bound to fluorescently labeled beads was incubated for 1 hour with an anti-ITGA11 antibody and mouse C2C12 cells that expressed human ITGA11 in binding medium containing DMEM, 10% normal goat serum and 2% normal rabbit serum. The cells were trypsinized and unbound collagen (type I rat tail collagen, Corning Labs) coated beads (fluorescent YG carboxylated Microspheres 2 micron (Polyscienced) were washed away from the cells 2× in PBS, resuspended in ice-cold PBS containing 2% fetal bovine serum, 1 mM EDTA, and the number of bound beads counted by flow cytometry. The results, shown in FIG. 1, FIG. 2 and FIG. 3 are expressed as the percentage of beads bound relative to the total number of beads incubated with the cells. MOPC 21 mAbIgG1 was used as a negative control.

Anti-ITGA11 antibodies FIB-918-1, FIB-918-2, FIB-918-3 and FIB-918-4 (FIG. 1); FIB-918-5, FIB-918-6, FIB-918-7, and FIB-918-8 (FIG. 2); and FIB-918-9 and FIB-918-10 (FIG. 3) inhibited binding of collagen to ITGA11 expressed in cells to differing degrees based on the range of IC50 observed.

### OTHER EMBODIMENTS

While the disclosure has been described in connection with specific embodiments thereof, it will be understood that it is capable of further modifications and this application is intended to cover any variations, uses, or adaptations of the disclosure following, in general, the principles of the disclosure and including such departures from the disclosure that come within known or customary practice within the art to which the disclosure pertains and may be applied to the essential features hereinbefore set forth, and follows in the scope of the claims. Other embodiments are within the claims.

1. An antibody or an antigen-binding fragment thereof that binds specifically to integrin α11 (ITGA11), wherein the antibody or antigen-binding fragment thereof comprises:
a complementarity-determining region (CDR) heavy chain 1 (CDR-H1) comprising the amino acid sequence of GFTFSSYA (SEQ ID NO: 1), GFTFSNAW (SEQ ID NO: 9), GFTFSSYS (SEQ ID NO: 14), GYTFTDYY (SEQ ID NO: 28), GFTFSDYW (SEQ ID NO: 36), or GFMFDTHA (SEQ ID NO: 46);
a complementarity-determining region (CDR) heavy chain 2 (CDR-H2) comprising the amino acid sequence of ISGSGGST (SEQ ID NO: 2), ISSSSSTI (SEQ ID NO: 15), FDPEDGET (SEQ ID NO: 29), or ISGSGGSI (SEQ ID NO: 74);
a complementarity-determining region (CDR) heavy chain 3 (CDR-H3) comprising the amino acid sequence of AKDLDWSGHDAFDI (SEQ ID NO: 3), ARDRGYSYSETSNDAFDI (SEQ ID NO: 10), ARGPDLSDYFDY (SEQ ID NO: 16), AKDPRGSGRDDAFDI (SEQ ID NO: 20), AKDPTTMTTDAFDI (SEQ ID NO: 25), ATLDYRGVVYFDY (SEQ ID NO: 30), AKDLLWAARDAFDI (SEQ ID NO: 37), AKQTVTSADDYFDY (SEQ ID NO: 43), ARSGETAGTDYFDY (SEQ ID NO: 48);
a complementarity-determining region (CDR) light chain 1 (CDR-L1) comprising the amino acid sequence of QSISSY (SEQ ID NO: 4), QTIGSY (SEQ ID NO: 21), SGSIASNY (SEQ ID NO: 31), QGINDF (SEQ ID NO: 40), QSVSSSY (SEQ ID NO: 49);
a complementarity-determining region (CDR) light chain 2 (CDR-L2) comprising the amino acid sequence of AAS (SEQ ID NO: 5), GAS (SEQ ID NO: 22), or EDK (SEQ ID NO: 32); and
a complementarity-determining region (CDR) light chain 3 (CDR-L3) comprising the amino acid sequence of QQTYSTPLT (SEQ ID NO: 6), QQSYSTPFT (SEQ ID NO: 11), QQSYSTPLT (SEQ ID NO: 17), QSYDSSNHWV (SEQ ID NO: 33), or QQDYNSPYT (SEQ ID NO: 50).

2. The antibody or antigen-binding fragment thereof of claim 1, wherein the CDR-H1 comprises the amino acid sequence of GFTFSSYA (SEQ ID NO: 1).

3. The antibody or antigen-binding fragment thereof of claim 1 or 2, wherein the CDR-H2 comprises the amino acid sequence of ISGSGGST (SEQ ID NO: 2).

4. The antibody or antigen-binding fragment thereof of any one of claims 1-3, wherein the CDR-L1 comprises the amino acid sequence of QSISSY (SEQ ID NO: 4).

5. The antibody or antigen-binding fragment thereof of any one of claims 1-4, wherein the CDR-L2 comprises the amino acid sequence of AAS (SEQ ID NO: 5).

6. The antibody or antigen-binding fragment thereof of any one of claims 1-5, wherein the CDR-L3 comprises the amino acid sequence of QQSYSTPFT (SEQ ID NO: 11).

7. The antibody or antigen-binding fragment thereof of claim 1, wherein the antibody or antigen-binding fragment thereof comprises:
a CDR-H1 comprising the amino acid sequence of GFTFSSYA (SEQ ID NO: 1);
a CDR-H2 comprising the amino acid sequence of ISGSGGST (SEQ ID NO: 2);
a CDR-H3 comprising the amino acid sequence of AKDLDWSGHDAFDI (SEQ ID NO: 3);
a CDR-L1 comprising the amino acid sequence of QSISSY (SEQ ID NO: 4);
a CDR-L2 comprising the amino acid sequence of AAS (SEQ ID NO: 5); and
a CDR-L3 comprising the amino acid sequence of QQTYSTPLT (SEQ ID NO: 6).

8. The antibody or antigen binding fragment thereof of claim 7, comprising:
a heavy chain variable domain comprising an amino acid sequence with at least 90% sequence identity to the amino acid sequence of SEQ ID NO: 7; and
a light chain variable domain comprising an amino acid sequence with at least 90% sequence identity to the amino acid sequence of SEQ ID NO: 8.

9. The antibody or antigen binding fragment thereof of claim 8, comprising:
a heavy chain variable domain comprising the amino acid sequence of SEQ ID NO: 7; and
a light chain variable domain comprising the amino acid sequence of SEQ ID NO: 8.

10. The antibody or antigen-binding fragment thereof of claim 1, wherein the antibody or antigen-binding fragment thereof comprises:
a CDR-H1 comprising the amino acid sequence of GFTFSNAW (SEQ ID NO: 9);
a CDR-H2 comprising the amino acid sequence of ISGSGGST (SEQ ID NO: 2);
a CDR-H3 comprising the amino acid sequence of ARDRGYSYSETSNDAFDI (SEQ ID NO: 10);
a CDR-L1 comprising the amino acid sequence of QSISSY (SEQ ID NO: 4);
a CDR-L2 comprising the amino acid sequence of AAS (SEQ ID NO: 5); and
a CDR-L3 comprising the amino acid sequence of QQSYSTPFT (SEQ ID NO: 11).

11. The antibody or antigen binding fragment thereof of claim 10, comprising:
a heavy chain variable domain comprising an amino acid sequence with at least 90% sequence identity to the amino acid sequence of SEQ ID NO: 12; and
a light chain variable domain comprising an amino acid sequence with at least 90% sequence identity to the amino acid sequence of SEQ ID NO: 13.

12. The antibody or antigen binding fragment thereof of claim 11, comprising:
a heavy chain variable domain comprising the amino acid sequence of SEQ ID NO: 12; and
a light chain variable domain comprising the amino acid sequence of SEQ ID NO: 13.

13. The antibody or antigen-binding fragment thereof of claim 1, wherein the antibody or antigen-binding fragment thereof comprises:
a CDR-H1 comprising the amino acid sequence of GFTFSSYS (SEQ ID NO: 14);
a CDR-H2 comprising the amino acid sequence of ISSSSSTI (SEQ ID NO: 15);
a CDR-H3 comprising the amino acid sequence of ARGPDLSDYFDY (SEQ ID NO: 16);
a CDR-L1 comprising the amino acid sequence of QSISSY (SEQ ID NO: 4);
a CDR-L2 comprising the amino acid sequence of AAS (SEQ ID NO: 5); and
a CDR-L3 comprising the amino acid sequence of QQSYSTPLT (SEQ ID NO: 17).

14. The antibody or antigen binding fragment thereof of claim 10, comprising:
a heavy chain variable domain comprising an amino acid sequence with at least 90% sequence identity to the amino acid sequence of SEQ ID NO: 12; and
a light chain variable domain comprising an amino acid sequence with at least 90% sequence identity to the amino acid sequence of SEQ ID NO: 13.

15. The antibody or antigen binding fragment thereof of claim 11, comprising:
a heavy chain variable domain comprising the amino acid sequence of SEQ ID NO: 12; and
a light chain variable domain comprising the amino acid sequence of SEQ ID NO: 13.

16. The antibody or antigen-binding fragment thereof of claim 1, wherein the antibody or antigen-binding fragment thereof comprises:
a CDR-H1 comprising the amino acid sequence of GFTFSSYA (SEQ ID NO: 1);
a CDR-H2 comprising the amino acid sequence of ISGSGGST (SEQ ID NO: 2);
a CDR-H3 comprising the amino acid sequence of AKDPRGSGRDDAFDI (SEQ ID NO: 20);
a CDR-L1 comprising the amino acid sequence of QTIGSY (SEQ ID NO: 21);
a CDR-L2 comprising the amino acid sequence of GAS (SEQ ID NO: 22); and
a CDR-L3 comprising the amino acid sequence of QQSYSTPFT (SEQ ID NO: 11).

17. The antibody or antigen binding fragment thereof of claim 16, comprising:
a heavy chain variable domain comprising an amino acid sequence with at least 90% sequence identity to the amino acid sequence of SEQ ID NO: 23; and
a light chain variable domain comprising an amino acid sequence with at least 90% sequence identity to the amino acid sequence of SEQ ID NO: 24.

18. The antibody or antigen binding fragment thereof of claim 15, comprising:
a heavy chain variable domain comprising the amino acid sequence of SEQ ID NO: 23; and
a light chain variable domain comprising the amino acid sequence of SEQ ID NO: 24.

19. The antibody or antigen-binding fragment thereof of claim 1, wherein the antibody or antigen-binding fragment thereof comprises:
a CDR-H1 comprising the amino acid sequence of GFTFSSYA (SEQ ID NO: 1);
a CDR-H2 comprising the amino acid sequence of ISGSGGST (SEQ ID NO: 2);
a CDR-H3 comprising the amino acid sequence of AKDPTTMTTDAFDI (SEQ ID NO: 25);
a CDR-L1 comprising the amino acid sequence of QSISSY (SEQ ID NO: 4);
a CDR-L2 comprising the amino acid sequence of AAS (SEQ ID NO: 5); and
a CDR-L3 comprising the amino acid sequence of QQSYSTPFT (SEQ ID NO: 11).

20. The antibody or antigen binding fragment thereof of claim 19, comprising:
a heavy chain variable domain comprising an amino acid sequence with at least 90% sequence identity to the amino acid sequence of SEQ ID NO: 26; and
a light chain variable domain comprising an amino acid sequence with at least 90% sequence identity to the amino acid sequence of SEQ ID NO: 27.

21. The antibody or antigen binding fragment thereof of claim 20, comprising:
a heavy chain variable domain comprising the amino acid sequence of SEQ ID NO: 26; and
a light chain variable domain comprising the amino acid sequence of SEQ ID NO: 27.

22. The antibody or antigen-binding fragment thereof of claim 1, wherein the antibody or antigen-binding fragment thereof comprises:
a CDR-H1 comprising the amino acid sequence of GYTFTDYY (SEQ ID NO: 28);
a CDR-H2 comprising the amino acid sequence of FDPEDGET (SEQ ID NO: 29);
a CDR-H3 comprising the amino acid sequence of ATLDYRGVVYFDY (SEQ ID NO: 30);
a CDR-L1 comprising the amino acid sequence of SGSIASNY (SEQ ID NO: 31);
a CDR-L2 comprising the amino acid sequence of EDK (SEQ ID NO: 32); and
a CDR-L3 comprising the amino acid sequence of QSYDSSNHWV (SEQ ID NO: 33).

23. The antibody or antigen binding fragment thereof of claim 22, comprising:
a heavy chain variable domain comprising an amino acid sequence with at least 90% sequence identity to the amino acid sequence of SEQ ID NO: 34; and
a light chain variable domain comprising an amino acid sequence with at least 90% sequence identity to the amino acid sequence of SEQ ID NO: 35.

24. The antibody or antigen binding fragment thereof of claim 23, comprising:
a heavy chain variable domain comprising the amino acid sequence of SEQ ID NO: 34; and
a light chain variable domain comprising the amino acid sequence of SEQ ID NO: 35.

25. The antibody or antigen-binding fragment thereof of claim 1, wherein the antibody or antigen-binding fragment thereof comprises:
a CDR-H1 comprising the amino acid sequence of GFTFSDYW (SEQ ID NO: 36);
a CDR-H2 comprising the amino acid sequence of ISGSGGST (SEQ ID NO: 2);
a CDR-H3 comprising the amino acid sequence of AKDLLWAARDAFDI (SEQ ID NO: 37);
a CDR-L1 comprising the amino acid sequence of QSISSY (SEQ ID NO: 4);
a CDR-L2 comprising the amino acid sequence of AAS (SEQ ID NO: 5); and
a CDR-L3 comprising the amino acid sequence of QQSYSTPFT (SEQ ID NO: 11).

26. The antibody or antigen binding fragment thereof of claim 25, comprising:
a heavy chain variable domain comprising an amino acid sequence with at least 90% sequence identity to the amino acid sequence of SEQ ID NO: 38; and
a light chain variable domain comprising an amino acid sequence with at least 90% sequence identity to the amino acid sequence of SEQ ID NO: 39.

27. The antibody or antigen binding fragment thereof of claim 26, comprising:
a heavy chain variable domain comprising the amino acid sequence of SEQ ID NO: 38; and
a light chain variable domain comprising the amino acid sequence of SEQ ID NO: 39.

28. The antibody or antigen-binding fragment thereof of claim 1, wherein the antibody or antigen-binding fragment thereof comprises:
a CDR-H1 comprising the amino acid sequence of GFTFSSYA (SEQ ID NO: 1);
a CDR-H2 comprising the amino acid sequence of ISGSGGST (SEQ ID NO: 2);
a CDR-H3 comprising the amino acid sequence of AKDLLWAARDAFDI (SEQ ID NO: 37);
a CDR-L1 comprising the amino acid sequence of QGINDF (SEQ ID NO: 40);
a CDR-L2 comprising the amino acid sequence of AAS (SEQ ID NO: 5); and
a CDR-L3 comprising the amino acid sequence of QQSYSTPLT (SEQ ID NO: 17).

29. The antibody or antigen binding fragment thereof of claim 28, comprising:
a heavy chain variable domain comprising an amino acid sequence with at least 90% sequence identity to the amino acid sequence of SEQ ID NO: 41; and
a light chain variable domain comprising an amino acid sequence with at least 90% sequence identity to the amino acid sequence of SEQ ID NO: 42.

30. The antibody or antigen binding fragment thereof of claim 29, comprising:
a heavy chain variable domain comprising the amino acid sequence of SEQ ID NO: 41; and
a light chain variable domain comprising the amino acid sequence of SEQ ID NO: 42.

31. The antibody or antigen-binding fragment thereof of claim 1, wherein the antibody or antigen-binding fragment thereof comprises:
a CDR-H1 comprising the amino acid sequence of GFTFSNAW (SEQ ID NO: 9);
a CDR-H2 comprising the amino acid sequence of ISGSGGST (SEQ ID NO: 2);
a CDR-H3 comprising the amino acid sequence of AKQTVTSADDYFDY (SEQ ID NO: 43);
a CDR-L1 comprising the amino acid sequence of QSISSY (SEQ ID NO: 4);
a CDR-L2 comprising the amino acid sequence of AAS (SEQ ID NO: 5); and
a CDR-L3 comprising the amino acid sequence of QQSYSTPFT (SEQ ID NO: 11).

32. The antibody or antigen binding fragment thereof of claim 31, comprising:
a heavy chain variable domain comprising an amino acid sequence with at least 90% sequence identity to the amino acid sequence of SEQ ID NO: 44; and
a light chain variable domain comprising an amino acid sequence with at least 90% sequence identity to the amino acid sequence of SEQ ID NO: 45.

33. The antibody or antigen binding fragment thereof of claim 32, comprising:
a heavy chain variable domain comprising the amino acid sequence of SEQ ID NO: 44; and
a light chain variable domain comprising the amino acid sequence of SEQ ID NO: 45.

34. The antibody or antigen-binding fragment thereof of claim 1, wherein the antibody or antigen-binding fragment thereof comprises:
a CDR-H1 comprising the amino acid sequence of GFMFDTHA (SEQ ID NO: 46);
a CDR-H2 comprising the amino acid sequence of ISGSGGSI (SEQ ID NO: 47);
a CDR-H3 comprising the amino acid sequence of ARSGETAGTDYFDY (SEQ ID NO: 48);
a CDR-L1 comprising the amino acid sequence of QSVSSSY (SEQ ID NO: 49);
a CDR-L2 comprising the amino acid sequence of GAS (SEQ ID NO: 22); and
a CDR-L3 comprising the amino acid sequence of QQDYNSPYT (SEQ ID NO: 50).

35. The antibody or antigen binding fragment thereof of claim 34, comprising:
a heavy chain variable domain comprising an amino acid sequence with at least 90% sequence identity to the amino acid sequence of SEQ ID NO: 51; and
a light chain variable domain comprising an amino acid sequence with at least 90% sequence identity to the amino acid sequence of SEQ ID NO: 52.

36. The antibody or antigen binding fragment thereof of claim 35, comprising:
a heavy chain variable domain comprising the amino acid sequence of SEQ ID NO: 51; and
a light chain variable domain comprising the amino acid sequence of SEQ ID NO: 52.

37. The antibody or antigen binding fragment thereof of claim 1, wherein the antibody or antigen-binding fragment thereof comprises a heavy chain variable domain comprising an amino acid sequence with at least 90% sequence identity to the amino acid sequence of any one of SEQ ID NOs: 7, 12, 18, 23, 26, 34, 38, 41, 44, or 51.

38. The antibody or antigen binding fragment thereof of claim 1 or 36, wherein the antibody or antigen-binding fragment thereof comprises a light chain variable domain comprising an amino acid sequence with at least 90% sequence identity to the amino acid sequence of any one of SEQ ID NOs: 8, 13, 19, 24, 35, 39, 42, 45, or 52.

39. The antibody or antigen-binding fragment thereof of any one of claims 1-38, wherein the antibody or antigen-binding fragment thereof is selected from the group consisting of a monoclonal antibody or antigen-binding fragment thereof, a polyclonal antibody or antigen-binding fragment thereof, a human antibody or antigen-binding fragment thereof, a humanized antibody or antigen-binding fragment thereof, a primatized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, a nanobody, an antibody-like protein scaffold, a domain antibody, a Fv fragment, a Fab fragment, a F(ab′)₂ molecule, and a tandem scFv (taFv).

40. The antibody or antigen-binding fragment thereof of claim 39, wherein the antibody or antigen-binding fragment thereof is a human, humanized, or chimeric antibody or antigen-binding fragment thereof.

41. The antibody or antigen-binding fragment thereof of any one of claims 1-38, wherein the antibody or antigen-binding fragment thereof binds specifically to a heterodimer of ITGA11 and ITGB1 (ITGA11β1 heterodimer).

42. A polynucleotide encoding the antibody or antigen-binding fragment thereof of any one of claims 1-41.

43. A vector comprising the polynucleotide of claim 42.

44. The vector of claim 43, wherein the vector is an expression vector.

45. The vector of claim 43, wherein the expression vector is a eukaryotic expression vector.

46. The vector of claim 45, wherein the vector is a viral vector.

47. The vector of claim 46, wherein the viral vector is selected from the group consisting of adenovirus (Ad), retrovirus, poxvirus, adeno-associated virus, baculovirus, herpes simplex virus, and a vaccinia virus.

48. A host cell comprising the vector of any one of claims 43-47.

49. A pharmaceutical composition comprising the antibody or antigen-binding fragment thereof of any one of claims 1-41, the polynucleotide of claim 42, the vector of any one of claims 43-47, or the host cell of claim 48, and a pharmaceutically acceptable carrier or excipient.

50. A kit comprising an agent selected from the antibody or antigen-binding fragment thereof of any one of claims 1-41, the polynucleotide of claim 42, the vector of any one of claims 43-47, the host cell of claim 48, or the pharmaceutical composition of claim 49.

51. A method of treating a subject having or at risk of developing a disorder, the method comprising administering to the subject the antibody or antigen-binding fragment thereof of any one of claims 1-41, the polynucleotide of claim 42, the vector of any one of claims 43-47, the host cell of claim 48, or the pharmaceutical composition of claim 49.

52. The method of claim 51, wherein the disorder is a fibrotic disorder.

53. The method of claim 52, wherein the fibrotic disorder is selected from hepatic fibrosis, kidney fibrosis, dermal fibrosis, heart fibrosis, vascular fibrosis, ocular fibrosis, bone marrow fibrosis, pulmonary fibrosis, glomerulonephritis, heart failure, scleroderma, excessive scar tissue post-surgery or device insertion, trauma or burns, progressive kidney disease, valvular heart disease, hypertensive heart disease, articular and periarticular fibrosis, myelofibrosis, ocular/vitreous fibrosis, intestinal fibrosis and stricture, peritoneal and retro-peritoneal fibrosis, pancreatic fibrosis, nephrogenic systemic fibrosis, and primary sclerosing cholangitis.

54. The method of claim 51, wherein the disorder is an inflammatory disorder.

55. The method of claim 54, wherein the inflammatory disorder is selected from asthma, airway inflammation, airway hyperreactivity, airway hyperresponsiveness, rhinosinusitis, rhinosinusitis with polyps, nasal polyposis, arthritis, eosinophilic inflammation, mast cell-mediated inflammatory diseases, sepsis, septic shock, seronegative enthesopathy and arthropathy (SEA) syndrome, osteoporosis, eosinophilic esophagitis, scleroderma, dermatitis, atopic dermatitis, allergic rhinitis, bullous pemphigoid, urticaria, cartilage inflammation, polymyalgia rheumatic, polyarteritis nodossa, Wegener's granulomatosis, Behcet's disease, myolitis, polymyolitis, dermatomyolitis, dermatomyositis, vasculitis, arteritis, diabetic nephropathy, interstitial cystitis, graft versus host disease (GVHD), gastrointestinal inflammatory conditions, and an inflammatory pulmonary condition.

56. The method of claim 51, wherein the disorder is a cancer.

57. The method of claim 56, wherein the cancer is selected from breast cancer, colorectal cancer, hepatic cancer, kidney cancer, liver cancer, lung cancer, pancreatic cancer, gastrointestinal cancer, melanoma, ovarian cancer, prostate cancer, cervical cancer, bladder cancer, glioblastoma, head and neck cancer, and cholangiocarcinoma.


SEQUENCE LISTING (52 sequences)

SEQ ID NO: 1 - protein, 8 aa, synthetic construct
GFTFSSYA

SEQ ID NO: 2 - protein, 8 aa, synthetic construct
ISGSGGST

SEQ ID NO: 3 - protein, 14 aa, synthetic construct
AKDLDWSGHDAFDI

SEQ ID NO: 4 - protein, 6 aa, synthetic construct
QSISSY

SEQ ID NO: 5 - skipped

SEQ ID NO: 6 - protein, 9 aa, synthetic construct
QQTYSTPLT

SEQ ID NO: 7 - protein, 121 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDLDWSGHDAFDIWGQGTTVTVSS

SEQ ID NO: 8 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQTYSTPLTFGGGTKVEIKR

SEQ ID NO: 9 - protein, 8 aa, synthetic construct
GFTFSNAW

SEQ ID NO: 10 - protein, 18 aa, synthetic construct
ARDRGYSYSETSNDAFDI

SEQ ID NO: 11 - protein, 9 aa, synthetic construct
QQSYSTPFT

SEQ ID NO: 12 - protein, 125 aa, synthetic construct
EVQLLESGGDLVKAGGSLRLSCAASGFTFSNAWMSWVRQAPGKGLEWVSGISGSGGSTYYADSVKGRFTISRDNSKNTLYLQMNSLRAEDTAVYYCARDRGYSYSETSNDAFDIWGRGTLVTVSS

SEQ ID NO: 13 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKLEIKR

SEQ ID NO: 14 - protein, 8 aa, synthetic construct
GFTFSSYS

SEQ ID NO: 15 - protein, 8 aa, synthetic construct
ISSSSSTI

SEQ ID NO: 16 - protein, 12 aa, synthetic construct
ARGPDLSDYFDY

SEQ ID NO: 17 - protein, 9 aa, synthetic construct
QQSYSTPLT

SEQ ID NO: 18 - protein, 119 aa, synthetic construct
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARGPDLSDYFDYWGRGTLVTVSS

SEQ ID NO: 19 - protein, 108 aa, synthetic construct
DIQLTQSPSSLSASVGDRVTITCRASQSISSYLNWYQQKPGKAPKLLIYAASSLQSGVPSRFSGSGSGTDFTLTISSLQPEDIATYYCQQSYSTPLTFGGGTKLEIKR

SEQ ID NO: 20 - protein, 15 aa, synthetic construct
AKDPRGSGRDDAFDI

SEQ ID NO: 21 - protein, 6 aa, synthetic construct
QTIGSY

SEQ ID NO: 22 - skipped

SEQ ID NO: 23 - protein, 122 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDPRGSGRDDAFDIWGQGTMVTVSS

SEQ ID NO: 24 - protein, 108 aa, synthetic construct
NIQMTQSPSSLSASVGDRVTITCRASQTIGSYLNWYQQKPGTAPKLLIYGASTSHTWVPSRFTGGGSGTEFTLTISSLQSEDFATYYCQQSYSTPFTFGGGTKLEIKR

SEQ ID NO: 25 - protein, 14 aa, synthetic construct
AKDPTTMTTDAFDI

SEQ ID NO: 26 - protein, 121 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDPTTMTTDAFDIWGQGTMVTVSS

SEQ ID NO: 27 - protein, 108 aa, synthetic construct; identical to SEQ ID NO: 13

SEQ ID NO: 28 - protein, 8 aa, synthetic construct
GYTFTDYY

SEQ ID NO: 29 - protein, 8 aa, synthetic construct
FDPEDGET

SEQ ID NO: 30 - protein, 13 aa, synthetic construct
ATLDYRGVVYFDY

SEQ ID NO: 31 - protein, 8 aa, synthetic construct
SGSIASNY

SEQ ID NO: 32 - skipped

SEQ ID NO: 33 - protein, 10 aa, synthetic construct
QSYDSSNHWV

SEQ ID NO: 34 - protein, 120 aa, synthetic construct
QVQLVQSGAEVKKPGATVKISCKVSGYTFTDYYMHWVRQAPGKGLEWMGGFDPEDGETIYAQKFQGRVTMTEDTSTDTAYMELSSLRSEDTAVYYCATLDYRGVVYFDYWGQGTLVTVSS

SEQ ID NO: 35 - protein, 112 aa, synthetic construct
NFMLTQPHSVSDSPGKTVTISCTGSSGSIASNYVQWYQQRPGSAPTTVIYEDKRRPSGVPDRFIGSIDSSSNSASLTISGLRTEDEADYYCQSYDSSNHWVFGGGTQLTVLG

SEQ ID NO: 36 - protein, 8 aa, synthetic construct
GFTFSDYW

SEQ ID NO: 37 - protein, 14 aa, synthetic construct
AKDLLWAARDAFDI

SEQ ID NO: 38 - protein, 121 aa, synthetic construct
EVQLVESGGGLVQPGGSLRLSCLASGFTFSDYWMAWVRQAPGKGLEWVSAISGSGGSTYYADSVKGRFTISRDNSKNTLYLQMNSLRAEDTAVYYCAKDLLWAARDAFDIWGQGTLVTVSS

SEQ ID NO: 39 - protein, 108 aa, synthetic construct; identical to SEQ ID NO: 13

SEQ ID NO: 40 - protein, 6 aa, synthetic construct
QGINDF

SEQ ID NO: 41 - protein, 121 aa, synthetic construct
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDLLWAARDAFDIWGQGTLVTVSS

SEQ ID NO: 42 - protein, 108 aa, synthetic construct
AIQLTQSPSTLSASVGDRVTITCRASQGINDFLAWYQQKPGKAPKLLIYAASSLQSGVPSRFSGSGSGTDFTLTISSLQPEDFATYYCQQSYSTPLTFGGGTKVEIKR

SEQ ID NO: 43 - protein, 14 aa, synthetic construct
AKQTVTSADDYFDY

SEQ ID NO: 44 - protein, 121 aa, synthetic construct
EVQLVESGGGVVRPGGPLRLSCAASGFTFSNAWMSWVRQAPGKGLEWVSAISGSGGSTYYADSVKGRFTISRDNSKNTLYLQMNSLRAEDTAIYYCAKQTVTSADDYFDYWGQGTLVTVSS

SEQ ID NO: 45 - protein, 108 aa, synthetic construct; identical to SEQ ID NO: 13

SEQ ID NO: 46 - protein, 8 aa, synthetic construct
GFMFDTHA

SEQ ID NO: 47 - protein, 8 aa, synthetic construct
ISGSGGSI

SEQ ID NO: 48 - protein, 14 aa, synthetic construct
ARSGETAGTDYFDY

SEQ ID NO: 49 - protein, 7 aa, synthetic construct
QSVSSSY

SEQ ID NO: 50 - protein, 9 aa, synthetic construct
QQDYNSPYT

SEQ ID NO: 51 - protein, 121 aa, synthetic construct
EVQLVESGGGLVQPGGSLRLSCAASGFMFDTHAMSWVRQAPGKGLEWVSSISGSGGSIYYADSVKGRFTISRDNSKNTLYLQMNSLRAEDTAVYYCARSGETAGTDYFDYWGQGTLVTVSS

SEQ ID NO: 52 - protein, 109 aa, synthetic construct
EIVMTQSPATLSLSPGERATLSCRASQSVSSSYLSWYQQKPGQAPRLLIYGASTRATGIPARFSGSGSGTDFTLTISSLQPEDFAVYYCQQDYNSPYTFGQGTKVDIKR